(19) United States
(12) Patent Application Publication
(10) Pub. No.: US 2005/0079492 A1
(43) Pub. Date: Apr. 14, 2005

(54) Micro-arrayed organization of transcription factor target genes

(76) Inventors: Robert M. Burgess Jr., San Diego, CA (US); Victoria Lunyak, La Jolla, CA (US); Leonid Noskin, Gatchina (RU)

Correspondence Address: Robert Burgess;TransGenetics Inc, Suite 134, 28835 Single Oak Drive, Temecula, CA, 92590, US

(21) Appl. No.: 10/275,845

(22) PCT Filed: Sep. 11, 2001
(86) PCT No.: PCT/US01/29048

Related U.S. Application Data
(60) Provisional application No. 60/232,075, filed on Sep. 12, 2000.

(51) Int. Cl. C12Q 1/68

(52) U.S. Cl. 435/6

(57) ABSTRACT

The following invention outlines methodologies for the construction and utilization of transcription factor direct target gene microarrays of both DNA and corresponding protein/peptide target origin. The technology entails the array/microarray annotation and organization of transcription factor direct loci and corresponding protein products identified through modified and improved versions of chromosomal immunoprecipitation (CHIP) and molecular cloning procedures. It allows for the formulation of physiologically directed arrays which result in a thorough, focused characterization of the genetic and biochemical regulation occurring within a give population of cells or a given tissue. Arrays and microarrays of direct targets for any given transcription factor created utilizing this technology are substantially more clinically relevant for purposes of medical diagnostics and patient prognostics than conventional microarrays due to the physiologically focused nature and the transcription factor targets. In addition, the characterization and array organization of transcription factor target protein products and the assessment of their interactions with other proteins and/or small molecules is of critical importance for the purposes of understanding cellular and ultimately the design of therapeutics for human anomalies.

### 1.0 FIELD OF THE INVENTION

The following invention describes the creation of array and microarray profiles of transcription factor targets for the purposes of physiologically focused medical diagnosis, patient prognosis and therapeutic development. It is accomplished through the utilization of modified and improved versions of the chromosomal immunoprecipitation (ChIP) assay and specific cloning methods combined with nucleotide and peptide/protein microarray technology to generate microarrays of transcription factor target gone and peptide sequences. These arrays allow for the efficient and saturable analysis of physiologically focused and restricted gene expression profiles and high-throughput biochemical screening of transcription factor drug target candidates for therapeutically relevant interacting molecules.

### 2.0 BACKGROUND OF THE INVENTION

Genetic activity, i.e. the activation or repression of gene transcription, has long been directly correlated with gene function. Transcriptional regulation is the first and perhaps most crucial mechanism by which cells regulate the functions of genes. By providing or denying mRNA templates for translation it is possible to tightly control the intricate cellular mechanisms of determination, division, survival etc. (FIG. 1 and for review see Moroy et al., 2000, Cellular and Molecular Life Sciences, 57(6): 957-75). Recently, a number of methods have been developed which allow for the rapid assessment of gene expression in a given sample and thus give insight as genetic profiles for various aspects of physiology and disease. These include, but are not limited to, two dimensional arrays and microarrays of either cDNAs or oligonucleotides representing corresponding mRNAs on solid supports. The arrayed aspect of the technology provides an organized, unbiased method for determining the quantitative and qualitative aspects of gene expression in a given sample population in a massive high-throughput format (a representative set of examples includes U.S. Pat. Nos. 6,136,592, 6,100,030, 6,040,138 herein incorporated by reference Debouck et al., 1999, Nature Genetics Supplement, 21: 48-50). It is this macromolecular ability to monitor the expression patterns and levels of genes involved in physiology and disease which allows for many basic science as well as clinical applications such as the assessment of predisposition to particular disorders as well as the possibility of disease prevention or early treatment.

It is clear that array technology enables researchers to efficiently ascertain expression patterns and levels of a multitude of loci within a particular sample. In addition, some effort has been directed towards the construction of microarrays which contain templates organized by physiology or functional entity such as cell cycle control or tissue specificity, yet these “focused arrays” are considerably lacking in gene content and limited in number. In addition, it still remains that the majority of genetic microarrays consist of random sequences, the identity and composition of which are often even unknown. Thus, for the most part, arrayed templates of either a nucleotide or peptide origin have yet to be developed such that the array of genes itself depicts something about physiology. It is therefore imperative that more focused, biologically relevant arrays and microarrays of genes be created. For example, arrays of genes known or hypothesized to be involved in a particular disease such as cancer, for example, would be of much more relevance clinically than arrayed organization of random gene sequences. By clustering arrays and microarrays in the context of specific physiologic and disease categories, these arrays can then be more readily subjected to the appropriate sample populations for analysis. This prevents the endless costly analysis of expression data which very well may not be relevant to the sample being studied. Therefore, an initial establishment of clusters and “families” of genes predicted to play particular roles in physiology or disease, and subsequent organization of these clusters in an array and microarray format will allow for a new level of discrete and focused genetic profiling for basic science and medical diagnostics.

One method for clustering genes into particular physiologic and disease categories relies upon the exploitation of either the direct or indirect interaction between transcriptional regulators and terminal target genes (for review see Tjian and Maniatis, 1994, Cell, 77: 5-8). Many transcription factors have been extensively demonstrated to play specific roles in very “focused” areas of physiology and disease, primarily through the regulation of target genes. It is possible to exploit this knowledge for the creation and production of functionally relevant arrays. By establishing arrays and microarrays of transcription factor target loci it is possible to narrow the purpose of said arrays for the characterization of expression profiles for specific aspects of physiology.

In addition to transcription factor target genetic expression pattern profiling, it is clear that characterization of the biochemical interaction properties of transcription factor targets will enhance therapeutic discovery and development. The ability to characterize protein/protein, chemical/protein, small molecule/protein and enzymatic reaction interactions in a high-throughput and saturable format is of unparalleled value for the eventual design of therapeutic intervention strategies for the treatment of disease. In order to efficiently search for and analyze these types of interactions in a high-throughput yet sensitive format it is necessary to implement variations of array and microarray technology. A number of groups have begun to focus upon the organization of proteins and/or peptide and amino acid sequences in array and microarray formats similar to that for nucleotides sequences. Such an organization has been successfully implemented for the efficient identification of specific interactions between arrayed protein samples and other entities which include, but are not limited to, other proteins, enzymes, metals, sugars, oligosaccharides, chemical compounds, DNA and RNA molecules (a representative set of examples includes U.S. Pat. Nos. 5,591,646, 6,156,511, 5,834,318 herein incorporated by reference; MacBeath et al., 2000, Science, 289: 1760-1763 and for review see Emili et al., 2000, Nature Biotechnology, 18: 393-397). These arrays allow for the high-throughput sensitive and specific characterization of interactions between arrayed proteins and other molecules. Yet in order to fully take advantage of protein array technology it is necessary to focus its application to discrete realms of physiology and disease. By concentrating the identities of protein arrays on particular facets of biology a great deal of irrelevant biochemical screening and the costs associated with it can be eliminated. It is the modification and narrowing of protein array and microarray technology in the context of transcription factor target proteins which is described in the present invention. The creation and utilization of transcription factor target protein microarrays will allow for the high-throughput identification of small molecules, enzymes and other proteins which interact specifically with these targets. Such characterizations will reveal novel enzymatic modification of protein targets as well as protein/protein, protein/DNA, protein/RNA and protein/small molecule interactions. The resulting transcription factor target protein biochemical interaction data will enable researchers to more efficiently focus their efforts on specific aspects of human physiology and disease in order to optimize the design of novel therapeutic intervention strategies for particular human anomalies.

In order to create arrays and microarrays of transcription factor target genes and the corresponding target protein sequences, it is necessary to discover and isolate the target genes in a complete and saturable fashion, as the more target genes present in a defined array the more thorough and complete the assessment of the genetic profile for the sample being analyzed. The chromosomal immunoprecipitation (ChIP) assay has been developed previously as a method for the analysis and characterization of transcription factor and/or regulatory protein interactions with known target sequences (Solomon et al., 1988, Cell, 53: 937-947). Recent advances in this technology now make it possible to identify and establish both direct and indirect relationships between transcriptional regulatory proteins and known as well as unknown target loci. Optimized in a high-throughput format, it is now possible to manipulate regulatory protein/DNA interactions in order to “scan the genome” in search of genes involved in discrete, focused aspects of physiology and disease (PCT patent application serial number PCT/US01/24823, filed Aug. 14, 2000 and herein incorporated by reference). By combining both modified chromosomal immunoprecipitation/target gene cloning methodologies and array/microarray technology, the presently described invention allows for creation of gene expression and protein interaction analysis tools such as expression and function-restricted arrays of particular focused physiologic relevance. FIG. 2 illustrates the construction of transcription factor target nucleotide microarrays through an application of modified chromosomal immunoprecipitation procedures in combination with molecular cloning methodologies. FIG. 4 diagrams methodology for the construction and implementation of transcription factor target protein “nonliving” arrays. These arrays and microarrays eliminate random nucleotide and peptide sequence characterization and enhance the detailed analysis of physiologically directed expression and biochemical profiling.

Originally, in order to take advantage of the inherent ability of transcription factors to dictate the regulation of specific downstream target genes for purposes of target gene identification, technologies such as CHIP were developed to extract transcription factor/known target gene interactions from living cells and tissues (Solomon et al., 1988, Cell, 53: 937-947). This technology, however, was limited to the identification of only known transcription factor targets. More recently, the ChIP methodology has been significantly improved upon and implemented for the efficient high-throughput identification and characterization of actively transcribed transcription factor target genes of both known and unknown origin (PCT patent application serial number PCT/US01/24823, filed Aug. 14, 2000 and herein incorporated by reference). Yet in order to fully take advantage of the knowledge of transcription factor target sequences for the purposes of therapeutic development it is apparent that efficient methodologies must be developed and employed which will reveal the genetic activity and biochemical nature of these target loci. The herein described technology accomplishes these goals and further extends the value of transcription factor target gene identification at the biochemical level for purposes of therapeutic development.

### 3.0 SUMMARY OF THE INVENTION

The application of array and microarray technologies for purposes of assessing genetic as well as biochemical interaction profiles of sample populations has been considerably limited by the construction of both nucleotide and peptide or protein arrays which do not represent discrete aspects of physiology and disease. This lack of focus impairs the analysis of expression patterns by including a great deal of loci which are often not relevant to the particular sample being studied, thereby resulting in an unnecessary allocation of resources to nonrelevant gene expression and biochemical interaction analysis. In addition, significant costs are associated with large-scale microarrays as well as misdirected analysis of valuable limited sample sources. The presently described invention, based upon transcription factor function, circumvents these hindrances by allowing for the construction of physiologic and disease oriented arrays and microarrays. By focusing the creation and implementation of arrays and microarrays on transcription factor target genes and the corresponding proteins, the presently described invention achieves significantly concentrated and discrete genetic and biochemical profiling. Furthermore, the employment of protein arrays and microarrays for purposes of identifying protein/protein, protein/small molecule and enzymatic interactions is becoming increasing valuable for the high-throughput efficient analysis and characterization of potential avenues for therapeutic intervention. It is the discrete organization and annotation of protein amino acid sequences in a format which allows for rapid assessment of interacting partners which drives the rapid accumulation of biochemical information. Yet this organization is of limited value if the microarrayed proteins themselves are of limited utility with respect to the long-term goal of identifying therapeutics for the treatment of human anomalies. The presently described invention lends significant improvement to protein array and microarray technologies by narrowing the arrayed material in a physiological context. By arraying and microarraying proteins which are of known function and value due to their classification as specific transcription factor targets, it will be possible to considerably eliminate the analysis and characterization of irrelevant biochemical interactions. Such narrowing of focus streamlines the drug discovery process, resulting in the requirement of fewer resources and a significant increase in the inherent value of the interaction data obtained. Transcription factors such as p53, for example, are strategically chosen which have been previously demonstrated to play critical roles in certain aspects of disease and physiology (FIG. 1). In vivo cross-linkage of protein/DNA complexes is performed in cell lines expressing the factor of interest and immunoprecipitation of protein/chromosomal complexes is subsequently employed through the utilization of antibodies specific for the transcription factor being studied (Solomon et al., 1988, Cell, 53: 937-947). Cross-linkage is reversed and purified DNA fragments representing target genes for the factor of interest are subjected to gene sequence or corresponding protein microarray construction. The transcribed downstream target sequences represent the functionality of the transcription factor in question as they directly carry out its function with respect to physiology. The protein and peptide outputs for transcription factor target genes represent downstream biochemical effectors for transcription factor function and potentially encode therapeutic targets. The aforementioned nucleotide and peptide or protein sequences are arrayed on solid supports such as nylon membrane, plastic or glass chips or even in vivo (see “living” arrays described below) and utilized to monitor the expression and interaction profiles of samples in question.

In order to successfully generate complex, saturable arrays and microarrays for particular aspects of physiology, the chromosomal immunoprecipitation assay has been modified and optimized for the high-throughput identification of both known and unknown transcription factor target loci (FIG. 2, FIG. 4 and PCT Patent application serial number PCT/US01/24823, filed Aug. 14, 2000 and herein incorporated by reference). Improvements include preimmunoprecipitation-immunoprecipitation (“preIP-IP”) utilizing antibodies specific for basal transcriptional machinery, which results in preisolation of only actively transcribed genes thus significantly reducing the acquisition of background random sequences. Subsequent immunoprecipitation is conducted on isolated complexes with antibodies which recognize particular transcription factors involved in discrete aspects of physiology and disease. In addition, sequences are isolated proximal to the transcriptional initiation site which often include 5′ untranslated and coding regions. The ability to direct immunoprecipitation of protein/DNA complexes to only actively transcribed regions of the genome is accomplished in the present invention through the use of antibodies specific for the large subunit of RNA polymerase II, the central component of the basal transcriptional machinery (Chang et al., 1998, Clinical Immunology and Immunopathology, 89(1): 71-8). In addition, the use of antibodies conjugated to solid supports such as magnetic beads results in significant increases in yield and sensitivity, thus making high-throughput capability feasible (Dynal Corporation Technical Handbook, 1998, Biomagnetic Applications in Cellular Immunology). These solid supports aid in the retrieval of protein/DNA complexes during initial and subsequent immunoprecipitation procedures by providing a matrix for retrieval of complexed material. It is also stated that sequential immunoprecipitation may be performed in any order with the end result being decreased background random sequences and increased yield obtained.

Additionally, a further elimination of background random sequences is obtained through the employment of inverse polymerase chain reaction (1-PCR) utilizing oligonucleotides specific for the transcription factor binding site (Ochlnan et al., 1988, Genetics, 120(3): 621-623; PCT Patent application serial number PCT/US01/24823, filed Aug. 14, 2000 and herein incorporated by reference). Acquisition of PCR products obtained by this methodology strongly infers direct target identity as products will only be obtained upon successful PCR extension from the inherent transcription factor binding sites present within immunoprecipitated fragments. The combination of these novel technologies along with standard cloning procedures and the creation of arrays and microarrays of target sequences obtained allows for the discrete assessment of expression profiling for virtually any aspect of physiology or disease. The proposed strategy would be indispensable for correct diagnostic tracing of disease progression and ultimately therapeutic intervention.

One embodiment of the present invention includes arrays and/or microarrays of transcription factor target genes, for the purposes of focusing genetic expression profiling experiments to particular specific entities of physiology and disease.

An additional embodiment of the present invention includes the methodology utilized to create the physiology, cellular morphology and disease oriented nucleotide arrays and microarrays. Said methodology, described herein, includes chromosomal immunoprecipitation, double immunoprecipitation utilizing antibodies to the basal transcriptional machinery, solid phase separation technologies and inverse-PCR combined with standard molecular cloning methods.

Another embodiment of the present invention is the antibodies utilized to immunoprecipitate crosslinked protein/DNA complexes from intact cells and/or tissues for purposes of creating arrays of transcription factor target genes and ultimately transcription factor target proteins.

Yet another embodiment of the present invention includes antibodies conjugated to solid phase supports, such as but not limited to magnetic beads, for purposes of increasing the yield of DNA template obtained and/or reducing the background of nonspecific random sequences obtained, for the further purposes of creating arrays and microarrays of transcription factor target genes.

Another embodiment of the present invention includes protein/DNA complexes isolated by modified ChIP methodologies described herein, for purposes of creating arrays and microarrays of transcription factor target genes.

Still another embodiment of the present invention includes DNA fragments isolated by the methodology described herein, for the purposes of creating arrays and microarrays of transcription factor target genes.

An additional embodiment of the present invention includes the nucleotide sequences corresponding to the transcription factor target genes identified by the methodology described herein, for purposes of creating physiologically and disease focused arrays and microarrays of transcription factor target genes.

Still another embodiment of the present invention includes the genetic profile information gleaned from application of transcription factor target nucleotide arrays and microarrays. It is this information which provides valuable insight with respect to particular realms of physiology and disease.

Yet another embodiment of the present invention is the application of transcription factor target gene sequence arrays and microarrays for purposes of medical diagnostics and patient prognostics.

Another embodiment of the present invention entails the peptide and amino acid sequences of the transcription factor target proteins which are organized and annotated in a microarrayed fashion. It is these sequences which are analyzed for interactions with other proteins, nucleotide sequences and chemical small molecule entities.

Yet another embodiment of the present invention includes the methodology for constructing transcription factor target protein arrays. It is the combination of modified chromosomal immunoprecipitation and molecular cloning and protein translation methods with biochemical array technology which results in the creation of valuable array reagents for therapeutic discovery.

An additional embodiment of the present invention includes “living”/biological arrays of transcription factor target proteins, for example, in the context of yeast colonies grown in a multiwel format which express the transcription factor target protein of interest. Living arrays allow for the characterization of interactions with the protein of interest in a biological context in which other components or factors may be required and thus provided by the yeast machinery to catalyze interactions with arrayed transcription factor target proteins.

Yet another embodiment of the present invention includes “nonliving”/chemical arrays and microarrays of transcription factor target proteins, for example, in the context of amino acid sequences bound either covalently or noncovalently to membranes or glass microchips.

An additional embodiment of the present invention includes the proteins, metals, small molecules and nucleotide sequences which are tested for interaction specificities with transcription factor target protein arrays and microarrays.

Yet another embodiment of the present invention includes the knowledge obtained from protein microarray studies revealing specific interaction data on transcription factor target proteins and their interactions with other proteins, enzymes or small molecule chemicals. It is the rapid accumulation of transcription factor target protein/protein and protein small molecule interaction data that will result in significant improvements in the efficiency and success of therapeutic development.

Still another embodiment of the present invention includes therapies developed as a result of knowledge obtained from the construction and implementation of transcription factor target protein arrays and microarrays.

### 4.0 DESCRIPTION OF THE FIGURES

FIG. 1 Is a diagrammatic illustration of transcriptional regulation by the tumor suppressor protein p53.

FIG. 2 Is an illustrative flowchart representing the manufacturing and construction of transcription factor target loci nucleotide microarrays for the purposes of medical diagnostics and patient prognostics (see text for details).

FIG. 3 Is a proposed example of an application of microarrayed p53 targets to the analysis of a particular sample as it progresses temporally from a normal to a tumorigenic cancerous phenotype and upon administration of different therapeutic strategies (see text for details).

FIG. 4 Is a diagrammatic illustration of the process of constructing and utilizing transcription factor target protein arrays and microarrays to determine target protein interacting molecules of either a chemical or biological nature (see text for details).

FIG. 5 Is a diagrammatic representation of the utilization of a “nonliving”/chemical transcription factor target protein microarray for the purposes of defining interacting molecules and the organization of data obtained into a database format (see text for details).

FIG. 6 Is a diagrammatic representation of the utilization of “living”/biological transcription facto target protein arrays for the purposes of defining interacting proteins, enzymes etc. in the context of yeast (see text for details).

FIG. 7 Illustrates the implementation of transcription factor target protein arrays for the discovery and development of cancer therapeutics by focusing on the biochemical properties of targets for the transcription factor p53 (see text for details).

Table 1 Is an example of transcription factor target gene microarray expression pattern data accumulated in a numerical format (see text for details).

Table 2 Is an example of the combination of phenotypic and environmental influences on genetic expression patterns depicted in a microarrayed numerical format (see text for details).

### 5.0 DETAILED DESCRIPTION OF THE INVENTION

5.1 Expression Analysis: The Development of Nucleotide Microarrays

Organized, large-scale analysis of expression patterns within given tissue or cell population samples has only recently become feasible. The ability to monitor the expression patterns of large numbers of genes and thus obtain a “genetic profile” of virtually any particular sample at any given timepoint promises to reveal in great detail molecular clues to physiology and disease. Indeed, known as “Transcriptomics,” this field is rapidly emerging as an essential and integral subdivision of the field of functional genomics (Drysdale et al., 2000, Yeast, 17(2):159-66).

A number of technologies have matured which allow for the organized annotation of genes for large-scale expression profiling purposes. These currently include the use of photochemical or inkjet technologies to array either cDNA or oligonucleotide sequences on solid supports such as glass slides or nylon membranes (INSERT MICROARRAY PATENT REFS HERE DeRisi et al., Science, 278: 680-686). It is predicted that eventually all genes from multiple organisms will be microarrayed for purposes of expression profiling of virtually any sample RNA population. Indeed, the entire compilation of loci present in the yeast genome has already been organized into a microarray format and said arrays have been proven to reveal functional genomics information in a highly reproducible manner (Spellman et al., 1998, Cell, 9: 3273-3297). The analysis of expression patterns and levels utilizing microarrays involves relatively straightforward recording of light emissions. Nucleotide microarrays are analyzed primarily for changes in expression via altered light wavelengths upon binding of sample RNA to cDNA or oligonucleotide sequences. The more bound RNA within a particular sequence slot present within the array, the brighter the emission of light and the greater the change in wavelength. Expression levels can therefore be accurately monitored with extreme sensitivity. In addition, given the micro aspect of the technology, relatively small sample populations can be analyzed for the actual character of the “transcriptome.”

The application of nucleotide microarray technology for purposes of monitoring gene expression levels and patterns is clear. From a basic science perspective, it is now possible to characterize changes in genetic expression patterns within a given tissue or cell line due to mutation or changes in environmental stimuli, for example. From a medical perspective, disease diagnosis and prognosis will benefit enormously from microarray technology. Monitoring gene expression patterns will result in the ability to diagnose predisposition to a certain disorder prior to its manifestation, and will allow doctors a head start on prevention and/or treatment. Yet, as discussed, current nucleotide microarray technology fails to organize and annotate subsets of loci which are specific for particular realms of physiology and disease. The presently described invention addresses this issue as well as the problems relating to it and provides a streamlined, high-throughput mechanism for the construction of physiologically specific microarrays of transcription factor target genes.

5.2 Protein Arrays and Microarrays

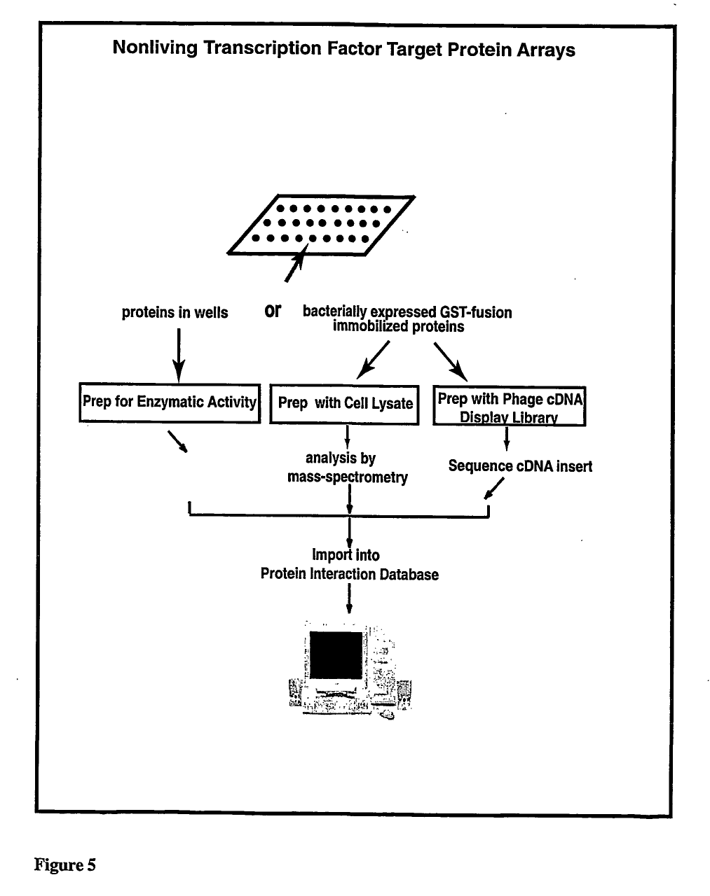

More recently, array and microarray technology has been developed for the characterization of protein/protein and protein/small molecule interactions. Specifically, methodologies have been developed which attach synthetic peptide and/or amino acid sequences corresponding to particular proteins to solid matrices with such sequences exposed on the surface of the matrix for purposes of accessibility by molecules of various origins which may or may not directly interact (MacBeath et al., 2000, Science, 289: 1760-1763). These “nonliving”/chemical arrays provide high-throughput characterization of direct interactions between organized, annotated proteins and/or peptides and other proteins or small molecules including metals, oligosaccharides and nucleotide sequences (FIG. 5). The technology is dependent upon the ability of these molecules to interact directly, however, without the requirement of other cofactors or modifications of the arrayed proteins which might be provided by living cells (Uetz et al., 2000, Nature, 403: 623-627).

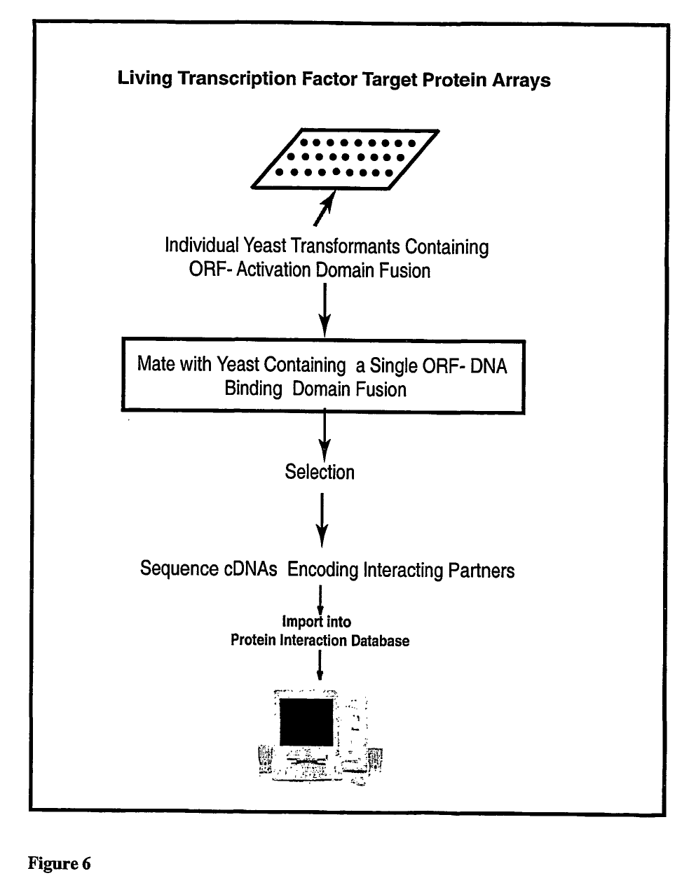

“Living”/biological arrays have also been developed which provide the opportunity for the modification of either the arrayed proteins or putative interacting proteins by the eukaryotic cellular machinery. Such modification or even the addition of other cellular components may be required for specific interaction between arrayed proteins and either small molecules or other proteins which are to be tested on the arrays. Living arrays have been successfully formulated in the context of the yeast strain S. cerevisiae, although others including those of high eukaryotic or bacterial origin may be constructed. In addition, however, protein arrays lack the focus necessary to efficiently scan for interacting molecules related to subsets of human physiology. As an example, arrayed clones of yeast are propagated in minimal media such that DNA sequences encoding open reading frame/GALA activation domain fusion proteins are translated in each prospective yeast colony. Interactions screens are performed by mating these arrayed yeast clones with another carrying ORF/GALA DNA binding domain fusion proteins. Survival of these colonies in a minimal media environment is dependent upon the interaction of these proteins and subsequent recruitment to a GAL4 DNA binding site upstream of a minimal promoter driving synthesis of an essential amino acid which is lacking in the minimal media context (FIG. 6). This strategy and others which include calorimetric assays in yeast have proven successful in identifying living arrayed protein interactions (Uetz et al., 2000, Nature, 403: 623-627).

5.3 Gene Expression and Function

Over the past 10 years enormous efforts have been focused on the sequencing, either partial or full length, and annotation of libraries of actively transcribed genes from limitless sources originating from countless organisms (for review see Zweiger et al., 1997, Trends in Biotechnology, 17: 429436). Recent advances in sequence database development have resulted in complex organization and annotation of known sequences into extensive gene families. Often these families demonstrate considerable conservation in sequence, and surprisingly expression pattern identity between organisms as diverse as fly and man. The gene encoding the transcription factor Nk×2.1 in humans, for example, is orthologous to the tinman locus in flies and each exibits similar roles in heart development for both fly and man (Chen et al., 1996, Developmental Genetics, 19(2): 119-30). This is but one example whereby both sequence composition as well as expression pattern give insight as to genetic function.

The utility of studying gene expression patterns and correlating these patterns to genetic and/or genomic function has become standard. Recent work in the analysis of yeast gene function suggests that the function of a particular locus or group of loci can be rapidly and accurately assessed by employing microarray technology to monitor changes in expression patterns of affected genes following mutation of particular loci. In addition, the same group has demonstrated the applicability of microarray analysis of gene expression to the study of pharmacological target validation (Hughes et al., 2000, Cell, 102: 109-126). Given these exciting results in yeast it is tempting to speculate how both nucleotide and protein/peptide array technology might be applied to the characterization of expression patterns in human cells and tissues. Yet the enormous complexity of the human genome requires a much more directed and focused approach to microarray construction, implementation and analysis and poses unique problems and issues which the presently described invention seeks to overcome.

5.4 High-Throughput Identification of Transcription Factor Targets

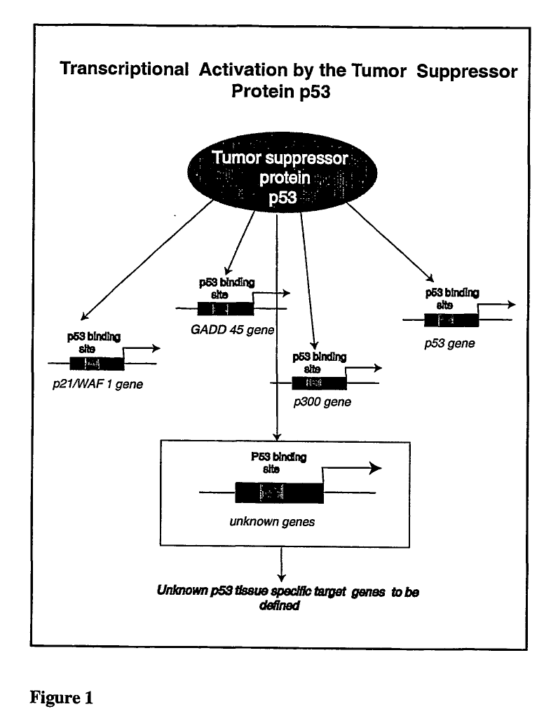

It is clear that transcription exemplifies function. Through tight regulatory cascades transcription factors direct unique symphonies of gene expression which constantly change with respect to environmental and temporal cues. A number of transcription factors have been characterized as functioning within a tight range with respect to physiology. That is, transcription factors often focus function on specific physiologic entities, most often through the activation or repression of target gene expression (for a review focused on pituitary organogenesis see Rhodes et al., 1994, Current Opinions in Genetic Development, 4: 709-717). Factors such as the estrogen receptor and the tumor suppressor p53, for example, control both cellular proliferation as well as programmed cell death and have been demonstrated to play crucial roles in the manifestation of breast cancer through the activation or repression of terminal target genes (FIG. 1; Tenbaum et al. 1997, International Journal of Biochemistry and Cell Biology, 29: 1325-1341; Levine et al., 1991, Nature, 351: 453-456). Other factors play roles in regulating cellular fate through early steps in the determination of specific lineages during development. An example of this is evident in the functional characterization of the transcription factor ikaros, which controls B and T cell development during hematopoiesis (Nichogiannopoulou et al., 1998 Seminars in Immunology, 10: 119-125). Still other factors regulate the development and/or function of specific organs. Similar to that mentioned for Nk×2.5 and tinman mentioned above, the GATA family of transcription factors has been shown to play a variety of roles in regulating cardiac specific gene activity both pre- and postnatally (Herzig et al., 1997, Proceedings of the National Academy of Sciences, 94: 7543-7548).

It is therefore evident that a dissection of the genetic hierarchies and ultimately an identification of target genes for these and other transcription factors as well as the biochemical interacting partners for these targets will yield valuable insight as to the genetic profile of particular discrete aspects of physiology. In order to saturably identify and annotate in a microarray format transcription factor target genes and proteins of both known and unknown as well as direct and indirect origin, the presently described invention expands upon previously developed technology (PCT patent application serial number PCT/US01/24823, filed Aug. 14, 2000 and herein incorporated by reference) by organizing transcription factor target genes and the corresponding protein/peptide sequences into an annotated arrayed format for use in expression and biochemical profiling.

5.5 Construction of Transcription Factor Target Nucleotide Microarrays

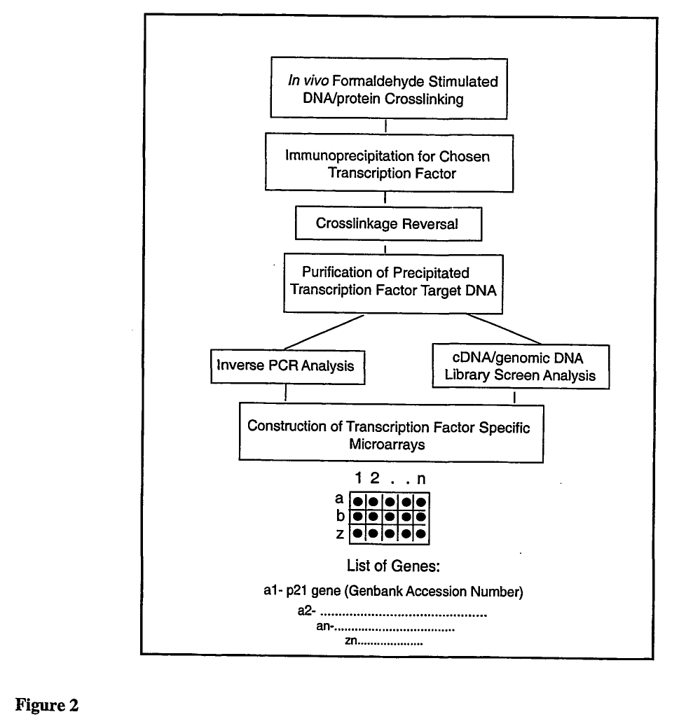

FIG. 2 illustrates the process for creation of transcription factor target nucleotide microarrays from manipulation of cell lines to final linkage of target sequences to two dimensional solid supports. As initial technology for the identification of transcription factor targets is described in detail in a previous patent application (FIG. 2 and U.S. Patent Ser. No. 60/225,225, filed Aug. 14, 2000 and herein incorporated by reference), it will only briefly be discussed herein. The process for construction of target microarrays initiates with the growth and expansion of appropriate cell lines expressing the transcription factor of interest, either endogenously or ectopically. Cell lines from which transcription factor target genes may be discovered via methodologies provided by the presently described invention include, but are in no way limited to 13C4 (mouse/mouse, hybrid, hybridoma), 143 B (human, bone, osteosarcoma), 2 BD4 E4 K99 (mouse/mouse, hybrid, hybridoma), 3 C9-D11-H11 (mouse/mouse, hybrid, hybridoma), 3 E 1 (mouse/mouse, hybrid, hybridoma), 34-5-8 S (mouse/mouse, hybrid, hybridoma), 3T3 (mouse, Swiss albino, embryo), 3T3 L1 (mouse, Swiss albino, embryo), 3T6 (mouse, Swiss albino, embryo), 5 C 9 (mouse/mouse, hybrid, hybridoma), 5G3 (hybrid, hybridoma), 6-23 (clone 6) (rat, thyroid, medullary, carcinoma), 7 D4 (mouse/rat, hybrid, hybridoma), 72 A1 (mouse/mouse, hybrid, hybridoma), 74-11-10 (mouse/mouse, hybrid, hybridoma), 74-124 (mouse/mouse, hybrid, hybridoma), 74-22-15 (mouse/mouse, hybrid, hybridoma), 74-9-3 (mouse/mouse, hybrid, B cells x myeloma, hybridoma, E cell), 76-7-4 (mouse/mouse, hybrid, hybridoma), 7C2C5C12 (mouse/mouse, hybrid, B cells x myeloma, hybridoma), 9 BG 5 (mouse/mouse, hybrid, hybridoma), 94-3 (mouse/mouse, hybrid, hybridoma), A 172 (human, glioblastoma), A 375 (human, malignant melanoma), A 72 (dog, golden retriever, connective, not defined tumor), A-427 (human, Caucasian, lung, carcinoma), A-498 (human, kidney, carcinoma), A-704 (human, kidney, adenocarcinoma), A549 (human, lung, carcinoma), ACHN (human, Caucasian, kidney, adenocarcinoma), ACT 1 (mouse/mouse, hybrid, hybridoma), AE1 (mouse/mouse, hybrid, hybridoma), AE-2 (mouse/mouse, hybrid, hybridoma), Aedes albopictus (mosquito-Aedes albopictus, larvae), AGS (human, Caucasian, stomach, adenocarcinoma), AK-D (cat, lung, embryonic), Amdur II (human, Caucasian, skin, fibroblast, methylmalonicacidemia), AV 3 (human, amnion), B 95.8 (monkey, marmoset, leukocyte), B-63 (mouse, mammary gland, carcinoma), B2-1 (mouse, BALB/c, embryo), B50 (rat, nervous system, nervous tissue glial tumor), B69 (mouse/mouse, hybrid, hybridoma), B95a (monkey, marmoset), BAE (bovine, aorta), BALB 3T12-3 (mouse, BALB/c, embryo), BALB 3T3 clone A31 (mouse, BALB/c, embryo), BB (fish—Ictalurus nebulosus (bulhead brown catfish), trunk), BBM.1 clone E9 (mouse/mouse, hybrid, hybridoma), BC3H1 (mouse, brain, brain tumor), BCE C/D-1b (bovine, cornea), BeWo (human, placenta, choriocarcinoma), BF-2 (fish—bluegill fry, caudal trunk), BGM (monkey, African green, kidney), BEK 21 clone 13 (hamster, golden Syrian, kidney), BNL CL.2 (mouse, BALB/c, liver, embryonic), BNL SV A.8 (mouse, liver, embryonic), BS/BEK (bovine, kidney, embryonic), BSC-1 (monkey, African green, kidney), BT (bovine, turbinate), Bu (MR-31) (buffalo, lung), BUD-8 (human, Caucasian, skin, fibroblast), BXPC-3 (human, pancreas, adenocarcinoma), C 1271 (mouse, R1, mammary gland, mammary tumor), C2C12 (mouse, muscle), C32 (human, melanoma, amelanotic), C 6 (rat, glial tumor), Caco-2 (human, Caucasian, colon, adenocarcinoma), Caki-1 (human, Caucasian, kidney, carcinoma), Caki-2 (human, Caucasian kidney, carcinoma), CaLu-1 (human, Caucasian, lung, carcinoma, epidermoid), Calu-3 (human, Caucasian, lung, adenocarcinoma), CAPAN 1 (human, Caucasian, pancreas, adenocarcinoma), CAPAN 2 (human, Caucasian, pancreas, carcinoma), CAR (fish—goldfish, fin), CCF-STTG1 (human, Caucasian, astrocytoma, anaplastic, grade IV), CCRF S 180 II (mouse, CFW, sarcoma), CCRF-CEM (human, Caucasian, peripheral blood, leukemia, acute lymphoblastic), CCRF-SB (human, Caucasian, peripheral blood, leukemia, acute lymphoblastic), CEM/C2 (human, leukemia, T cell), Cf2Th (dog, thymus), Chang liver (human, liver), CHO K1 (hamster, Chinese, ovary), CHP 3 (human, Black, skin, fibroblast, galactosemia), CHP 4 (human, Black, skin, fibroblast, asymptomatic galactosemia), CHSE 214 (fish—salmon, embryo), Clone 1-5c WKD of Chang Conjunctiva (human, conjunctiva), Clone M-3 (mouse, (CxDBA) F1, skin, melanoma), CMT 93 (mouse, C57BL/ICRFat, rectum, carcinoma), COS-1 (monkey, African green, kidney), COS-7 (monkey, African green, kidney), CPA (bovine, endothelium, pulmonary artery), CPA 47 (bovine, endothelium, pulmonary artery), CPAE (bovine, endothelium, pulmonary artery), CRFK (cat, domestic, kidney), CR1-D11 (rat, NEDH, insulinoma), CSE 119 (fish—salmon, embryo), CV 1 (monkey, African green, kidney), CVC 7 (Agrothis segetum, hybrid, hybridoma), D 17 (dog, bone, sarcoma, osteogenic), Daudi (human, Black, lymphoma, Burkitt), DB 9 G.8 (mouse/mouse, hybrid, hybridoma), DB 1-Tes (dolphin, Delphinus bairdi, testis), DeDe (hamster, Chinese, lung), Detroit 510 (human, Caucasian, skin, fibroblast, galactosemia), Detroit 525 (human, Caucasian, skin, fibroblast, Turner syndrome), Detroit 529 (human, Caucasian, skin, fibroblast, trisomy 21/Down syndrome), Detroit 532 (human, Caucasian, foreskin, trisomy 21/Down syndrome), Detroit 539 (human, Caucasian, skin, fibroblast, trisomy 21/Down syndrome), Detroit 548 (human, Caucasian, skin, fibroblast, partial D trisomy), Detroit 550 (human, skin, fibroblast), Detroit 551 (human, Caucasian, skin, embryonic), Detroit 562 (human, Caucasian, pharynx, carcinoma), Detroit 573 (human, Caucasian, skin, fibroblast, B/D translocation), Detroit 6 (human, bone marrow), DK (dog, beagle, kidney), DON (hamster, Chinese, lung), DU 145 (human, Caucasian, prostate, carcinoma), Duck embryo (duck, Pekin, embryo), E.Derm (horse, dermis), EBTr (bovine, trachea, embryonic), ECTC (bovine, thyroid, embryonic), ECV304 (human, Asiatic, umbilical cord), EIAV 12E8.1 (mouse/mouse, hybrid, hybridoma), Ep 16 (mouse/mouse, hybrid, hybridoma), EPC (fish, carp epidermal, epithelioma), EREp (rabbit, skin, embryonic), ESK4 (pig, kidney, embryonic), FBHE (bovine, heart, embryonic), Fc 2 Lu (cat, lung, embryonic), Fc 3 Tg (cat, tongue, embryonic), FeLV 3281 (cat, lymphoma), FHM (fish—minnow, skin), FL (human, amnion), FRhK4 (monkey, rhesus, kidney, embryonic), G-7 (mouse, Swiss-Webster, muscle), G.8 (mouse, Swiss-Webster, muscle), GCT (human, lung, metastasis, histiocytoma), GH 1 (rat, Wistar-Furth, pituitary tumor), GH 3 (rat, Wistar-Furth, pituitary tumor), Girardi heart (human, heart), GK 1.5 (mouse/rat, hybrid, hybridoma) H 16-L104R 5 (mouse/mouse, hybrid, hybridoma), H 9 (human, leukemia, acute lymphoblastic), H 4-H-E (rat, liver, hepatoma), H4 (human, Caucasian, brain, nervous tissue glial tumor), H4-II-E-C3 (rat, AxC, liver, hepatoma), H4TG (rat, liver, hepatoma), H-9c2(2-1) (rat, BDIX, heart), Hak (hamster, Syrian, kidney), HCT 116 (human, colon, carcinoma), HCT-8 (human, intestine, ileocecal, adenocarcinoma), HEL 299 (human, Caucasian, lung, embryonic), HeLa (human, Black, cervix, carcinoma, epitheloid), HeLa 229 (human, Black, cervix, carcinoma, epitheloid), HeLa S 3 (human, Black, cervix, carcinoma, epitheloid), Hep 2 (human, Caucasian, larynx, carcinoma, epidermoid), Hep 3B2.1-7 (human, liver, carcinoma, hepatocellular), Hep G2 (human, Caucasian, liver, carcinoma, hepatocellular), Hepa 1-6 (mouse, liver, hepatoma), HFL (human, lung), HG 261 (human, Caucasian, sldn, fibroblast, Fanconi anemia), HGP 24 (human, gingival stroma), HL 60 (human, Caucasian, peripheral blood, leukemia), HOS (human, Caucasian, bone, osteosarcoma), HR_{T} 18 (human, rectum-anus, adenocarcinoma), Hs 683 (human, neuroglia, glioma), Hs 863.T (human, bone, sarcoma, Ewing's), HS 883.T (human, bone, giant cell, sarcoma), HS 888 Lu (human: Caucasian, lung), Hs-27 (human, foreskin), HSDM1C1 (mouse, Swiss albino, fibrosarcoma), HT 1080 (human, Caucasian, acetabulum, fibrosarcoma), HT 1376 (human, Caucasian, bladder, carcinoma), HT-29 (human, Caucasian, colon, adenocarcinoma), HuTu 80 (human, adenocarcinoma), I 10 (mouse, BALB/cJ, testis, Leydig cells, testicular tumor), IB-RS-2 (pig, kidney), IBRS-2 D10 (pig, kidney), IEC-6 (rat, intestine, small), IM-9 (human, Caucasian, bone marrow, multiple myeloma), IMR 31 Bu (buffalo, lung), JMR 32 (human, Caucasian, neuroblastoma), IMR-90 (human, Caucasian, lung, embryonic), Intestine 407 (human, Caucasian, intestine, embryonic), J 111 (human, leukemia, monocytic), J 774A. 1 (mouse, BALB/c, monocyte-macrophage, not defined tumor), Jensen sarcoma (rat, sarcoma), JH 4 clone 1 (guinea pig, strain 13, lung), Jiyoye (human, Black, ascitic fluid, lymphoma, Burkitt), JM (human, leukemia, T cell), Jurka J6 (human, leukemia, T cell), K 562 (human, Caucasian, pleural effusion, leukemia, chronic myeloid), KATO III° (human, Mongoloid, stomach, carcinoma), KB (human, Caucasian, mouth, carcinoma, squamous cell), KHOS/NP (human, Caucasian, bone, osteosarcoma), KMP (mouse), L 1210 (mouse, ascitic fluid, leukemia, lymphocytic), L 132 (human, lung, embryonic), L 21.6 (mouse hybrid, hybridoma), L 243 (mouse/mouse, hybrid, hybridoma), L 5.1 (mouse/mouse, hybrid, hybridoma), L 929 (mouse, C3H (An, connective), L6 (rat, skeletal muscle), LC 540 (rat, Fisher, testis, Leydig cells, testicular tumor), LLC-MK2 (monkey, rhesus, kidney), LLC-PK1 (pig, kidney), LLC-RK1 (rabbit, New Zealand white, kidney), LLC-WRC 256 (rat, Walker, carcinoma), LM from NCTC clone 929 (mouse, C3H/An, connective), LM TK negative (mouse, C3H/An, connective), LNCaP.FGC (human, Caucasian, prostate, carcinoma), LS 180 (human, Caucasian, colon, adenocarcinoma), M 1 (mouse, SL, bone marrow, leukemia, myeloid), M-2E6 (mouse/mouse, hybrid, hybridoma), M2-1C₆₋₄R3 (mouse/mouse, hybrid, hybridoma), MA 104 (monkey, African green, kidney, embryonic), mAB 35 (mouse/rat, hybrid, B cells x myeloma, hybridoma, B cell), MARC 145 (monkey, kidney), Mc Coy (mouse), MC/CAR (human, plasmacytoma, B cell), MCF 7 (human, Caucasian, breast, adenocarcinoma), MDBK (bovine, kidney), MDBK (13U 100) (bovine, kidney), MDCC MSB 1 (chicken, avian, spleen, lymphoma), MDCK (dog, cocker spaniel, kidney), MDOK (sheep, kidney), MDTC RP 19 (turkey, lymphocyte, Marek's disease), MEL Im (monkey, rhesus, mammary gland, mammary tumor), MG-63 (human, bone, osteosarcoma), MH 1 C 1 (rat, buffalo, liver, hepatoma), MH-S (mouse, lung), MIA PaCa-2 (human, Caucasian, pancreas, carcinoma), MiCl1 (mustela vison (mink), lung), MK-D6 (mouse/mouse, hybrid, hybridoma), MLA 144 (gibbon, lymphosarcoma), MOLT-3 (human, peripheral blood, leukemia, acute lymphoblastic T cell), MOLT4 (human, peripheral blood, leukemia), MPC-11 (mouse, BALB/c, myeloma), MPK (minipig, kidney), MRC 5 (human, lung, embryonic), MRSS-1 (mouse/mouse, hybrid, hybridoma, T cell), MS (monkey), Mv 1 Lu (mustela vison (mink), lung), MVPK-1 (pig, kidney), NA C 1300 clone (mouse, brain, neuroblastoma), Namalwa (human, Black, lymphoma, Burkitt), NCTC 2544 (human, skin, keratinocyte), NCTC clone 3526 (monkey, rhesus, kidney), Neuro-2a (mouse, albino, neuroblastoma), NIH:OVCAR-3 (human, Caucasian, adenocarcinoma, ovary), NOR 10 (mouse, muscle), NRK 49F (rat, kidney), NSO (mouse, BALB/c, myeloma), OA1 (sheep, brain), OHH1.K (deer, kidney), OKT 3 (mouse/mouse, hybrid, hybridoma), OKIT 4 (mouse/mouse, hybrid, hybridoma), OKT 8 (mouse/mouse, hybrid, hybridoma), P 3 HR 1 human, lymphoma, Burkitt), P3 88 D1 (mouse, DBA/2, monocyte-macrophage, lymphoma), P3 NS1 Ag4 (mouse, myeloma), P3NP/PFN (mouse/mouse, hybrid, hybridoma), P815 (mouse, mastocytoma), PANC-1 (human, Caucasian, pancreas, carcinoma), PC 61-5-3 (mouse/rat, hybrid, hybridoma), PC-12 (rat, adrenal medulla, pheochromocytoma), PD 5 (pig, kidney), PEG 1-6 (mouse/mouse, hybrid, B cells x myeloma, hybridoma, B cell), PK 15 (pig, kidney), PLC/PRF/5 (human, liver, hepatoma, Alexander cells), Pt K1 (marsupial—potoroo, kidney), QT 35 (quail, Japanese, fibrosarcoma), QT 6 (quail, Japanese, fibrosarcoma), R 2 C (rat, Wistar-Furth, testis, Leydig cells, testicular tumor), R 9 ab (rabbit, New Zealand white, lung), R D (human, Caucasian, muscle, rhabdomyosarcoma, embryonal), R63 (mouse/mouse, hybrid, B cells x myeloma, hybridoma, B cell), RAB-9 (rabbit, New Zealand white, skin, fibroblast), Raji (human, Black, lymphoma, Burkitt), RBL 1 (rat, leukemia, basophilic), RFL 6 (rat, Sprague-Dawley, lung), RK 13 (rabbit, kidney), RK 13/1 (rabbit, kidney), RPMI 1788 (human, Caucasian, peripheral blood), RPMI 1846 (hamster, golden Syrian, skin, melanoma, melanotic), RPMI 2650 (human, nasal septum, carcinoma, squamous cell), RPMI 8226 (human, peripheral blood, myeloma), RR 1022 (rat, Amsterdam, sarcoma), RTG 2 (fish—trout rainbow, gonad), RTO (fish—trout, rainbow, ovary), Saos-2 (human, Caucasian, bone, osteosarcoma), Sf 1 Ep (rabbit, domestic, epidermis), SIRC (rabbit, cornea), SK-LU-1 (human, Caucasian, lung, adenocarcinoma, grade EII), SK-MES-1 (human, lung, carcinoma, squamous cell), SK-NEP-1 (human, Caucasian, kidney, Wilms' tumor), SK-OV-3 (human, Caucasian, ovary, adenocarcinoma), SSE 5 (fish—trout, embryo), STO (mouse, SIM, embryo), SV-T2 (mouse, BALB/c, embryo), SW 13 (human, Caucasian, adrenal cortex, adenocarcinoma), T 98 G (human, Caucasian, glioblastoma), Th 1 Lu (bat, lung), TE 671 (human, Caucasian, medulloblastoma), TK TS 13 (hamster, Syrian, kidney), U 937 (human, Caucasian, pleural effusion, lymphoma, histiocytic), VERO (monkey, African green, kidney), VERO 76 (monkey, African green, kidney), VERO C 1008 (monkey, African green, kidney), WC 1 (fish, dermis, sarcoma), WF 2 (fish—Walley whole fry, fibroblast), WI 26 VA 4 (human, Caucasian, lung, embryonic), WI 38 (human, Caucasiar lung, embryonic), WI 38 VA 13 (human, Caucasian, lung, embryonic), WI-1003 (human, lung), WISH (human, amnion), WM 115 (human, skin, melanoma), XC (rat, Wistar, sarcoma), Y 1 (mouse, LAF1, adrenal cortex, adrenal tumor), ZR-75-1 (human, Caucasian, breast, carcinoma) and any other as yet undiscovered or uncharacterized cell lines through which the presently described invention may be implemented for the discovery of transcription factor target genes. It is contemplated by the present invention that tissues of various sources may also be utilized for the purposes of constructing transcription factor target nucleotide and/or protein/peptide arrays. Tissues include, but are not limited to heart, brain, spleen, lung, liver, muscle, kidney, testis, ovary, gut, hypothalamus, pituitary, tooth bud, mesoderm, ectoderm, endoderm, neural tube, somite, smooth muscle, cardiac muscle, skeletal muscle and all embryonic tissues from all possible organisms and all possible timepoints.

Intact cells or tissues are treated with protein/DNA cross-linkage reagents such as formaldehyde as previously described. While the present invention employs formaldehyde as a chemical component for the cross-linking of protein/DNA complexes in living cells and tissues, it is in no way limited to this reagent for fixation. Other chemicals may also be utilized to fix proteins to DNA (Benashski et al., Methods, 2000, 22: 365-371). Some of these include, but are in no way limited to homobifunctional compounds difluoro-2,4-dinitrobenzene (DFDNB), dimethyl pimelimidate (DMP), disuccinimidyl suberate (DSS), thcarbodiimide reagent EDC, psoralens including 4,5′,8-trimethylpsoralen, photo-activatable azides such as ¹²⁵I(S-[2-(4-azidosalicylamido)ethylthio]-2-thiopyridine) otherwise known as AET, (N-[4(p-axidosalicylamido)butyl]-3′[2′-pridyldithio]propionamide) also known as APDP, the chemical cross-linking reagent Ni(II)-NH2-Gly-Gly-His-COOH also known as Ni-GGH, sulfosuccinimidyl 2. [(4-axidosalicyl) amino]ethyl]-1,3-dithiopropionate) also known as SASD, (N-14-(2-hydroxybenzoyl)-N-11 (4-azidobenzoyl)-9-oxo-8,11,14-triaza-4,5-ditheatetradecanoate) and any as yet uncharacterized or undiscovered reagents which result in the cross-linking of protein/DNA complexes in living cells and tissues.

Cellular extracts are purified and sonicated to yield the desired chromatin fragment size and said extracts are subjected to antibodies linked to solid phase supports such as M450 tosylactivated magnetic beads. Other magnetic beads contemplated by the present invention and created by Dynal Corporation which may be utilized as a solid phase support for the chromosomal immunoprecipitation reaction described herein include Dynabeads M450 uncoated, Dynabeads M-280 Tosylactivated, Dynabeads M450 Sheep anti-Mouse IgG, Dynabeads M450 Goat anti-Mouse IgG, Dynabeads M450 Sheep anti-Rat IgG, Dynabeads M450 Rat anti-Mouse IgM, Dynabeads M-280 sheep anti-Mouse IgG, Dynabeads M-280 Sheep anti-Rabbit IgG, Dynabeads M450 sheep anti-Mouse IgG1, Dynabeads M-450 Rat anti-Mouse IgG1, Dynabeads M450 Rat anti-Mouse IgG2a, Dynabeads M-450 Rat anti-Mouse IgG2b, Dynabeads M450 Rat anti-Mouse IgG3. Other magnetic beads which are also contemplated by the present invention as providing utility for the purposes of sequential immunoprecipitation include streptavidin coated Dynabeads.

While the presently described invention employs magnetic beads as the solid phase to increase yield and recovery of protein/DNA complexes during sequential chromosomal immunoprecipitation, it is in no way the only solid phase support system which may be implemented successfully to increase yield and sensitivity. Other solid phase supports contemplated by the present invention include, but are not limited to, sepharose, chitin, protein A cross-linked to agarose protein G cross-linked to agarose, agarose cross-linked to other proteins, ubiquitin cross-linked to agarose, thiophilic resin, protein G cross-linked to agarose, protein L cross-linked to agarose and an support material which allows for an increase in the efficiency of purification of protein/DNA complexes.

Antibodies specific for components of the basal transcriptional machinery and/or the transcription factor of interest recruit both the factor and bound potential target DNA sequences to the solid support matrix. A series of washing steps removes nonspecific background bound sequences. Cross-linkage is reversed and a heterogenous population of DNA templates putatively representing transcription factor target genes is retrieved. The implementation of molecular biological procedures including inverse-PCR (Ochman et al., 1988, Genetics, 120(3): 621-623) and cDNA library screening results in the isolation of transcribed sequences for each target gene as well as confirmation of direct target gene identity. Upon identification of transcription factor target loci, microarrays may subsequently be constructed which annotate and organize these target sequences into specific physiologically focused expression analysis tools based upon the original transcription factors immunoprecipitated in the modified sequential ChIP process. Transcription factor target sequences are attached to solid supports such as nylon membranes, glass or plastic chips in the form of either cDNAs or oligonucleotides. Although the presently described invention contemplates the use of nylon membranes as well as glass or plastic chips as solid phase supports it is in no way limited to these materials for the ultimate construction of transcription factor target nucleotide arrays. Other solid supports include, but are in no way limited to nitrocellulose and metals of any kind. A blueprint of each array documents the identity of each gene and its location relative to others on the two dimensional solid support. Said arrays and microarrays are hence subjected to appropriate tissue and cell samples to produce sample expression profiles. Hybridization of RNA or cDNA samples from test populations to the transcription factor target nucleotide arrays allows for sensitive expression profiling of particular realms of physiology. By narrowing the focus of each array and microarray to transcription factor target genes, these arrays serve specific purposes with respect to physiology, morphology and disease, and eliminate many of the disadvantages of large-scale whole genome and/or unfocused array technology.

The creation of both nucleotide and peptide or protein arrays and microarrays can be performed for a variety of tissue and cell type-specific transcription factors for the purposes of physiologically focused gene expression analysis and biochemical interaction characterization. While the presently described invention focuses on the discovery of both known and previously undiscovered target loci for the transcription factor p53 and the corresponding array construction for these targets, it is in no way limited in its utility for this particular transcription factor or the targets thereof. Other transcription factors and corresponding targets of prokaryotic, eukaryotic and viral origin contemplated and covered by the present invention include, but are not limited to A2, AAF, abaA abd-A, Abd-B, ABF1, ABF-2, ABI4, Ac, ACE2, ACF, ADA2, ADA3, ADA-NF1, Adf-1, Adf-2a, Adf-2b, ADR1, AEF-1, AF-1, AF-2, AFLR, AFP1, AFX-1, AG, AG1, AG2, AG3, AGIE-BP1, AGL11, AGL12, AGL13, AGL14, AGL15-1, AGL15-2, AGL17, AGL2, AGL3, AGILA, AGL6, AGL8, AGL9, AhR, AIC3, AIC2, AIC3, AIC4, AIC5, AID2, AIIN3, ALF1B, ALL-1, alpha. 1, alpha2uNF1, alpha2uNF2, alph2uNF3, alpha-CP1, alpha-CP2a, alpha-CP2b, alpha-factor, alphaH0, alphaH2, alphaH3, alpha-IRP, alpha-PAL, alpha2uNF1, alpha2uNP3, alphaA-CRYBP1, alphaH2-alphaE3, alphaMHCBF1, Alx-3, ALx4, ALY, AMDA, AmdR, aM-2, AML1, AMLia, AMLlb, AMLlc, AMLIDeltaN, AML2, AML3, AMT1, AMY-1L, A-Myb, AN2, AnCF, ANF, ANF-2, ANR1, Antp, AP-1, AP-2, AP-2alphaisoform2, AP-2alphaisoform3, AP-2alphaisoform4, AP-3, AP3-1, AP3-2, AP4, AP-5, APC, APERALA1, APETALA3, AR, ARA, AREA, AREB6, ARG R1, ARG R11, armadillo, Arnt, ARP-1, ARP7, ARP9, ARR1, AS-C T3, AS321, ASF-1, ASH-1, ASH-3b, ASP, AT-13P2, ATBF1-A, ATBP, AT-BP1, AT-BP2, ATF, ATF-1, ATF-3, ATF-3deltaZ1P, ATF-adelta, ATF-like, Athb-1, Athb-2, Ato, Axial, AZF1, B factor, B″, BAF1, B-TFIID, band I factor, BAP, Barx-1, BAS, BBF1, BBF2a, BBF3, BBFa, Bcd, BCF1, Bcl-3, BCL-6, BD73, BDF1, beta-1, BETA1, BETA2, beta-catenin, beta-factor, BF-1, BF-2, BGP1, Binl, Blimp-1, BmFTZ-F1, B-Myb, B-Myc, BP1, BP2, B-Peru, BR-C Z1, BR-C Z2, BR-C Z4, Brachyury, BRF1, BrIA, Bm-3a, Brn4, Brn-5, BUF1, BUF2, BAF1, BAS1, BCFII, beta-factor, BETA3, BLyF, BP2, BR-C Z3, brachyuray, brahma, BRF1, Brn1, Brn2, Bm-3a, Brn-3b, Brn-4, Brn-5, Bro, Btd, BTEB, BTEB2, BUF, BUF1, BUF2, BUR6, byr3, BZIP910, BIP911, c-abl, c-Ets-1, c-Ets-2, c-Fos, c-Jun, c-Maf, c-myb, c-Myc, c-Qin, c-Re1, C/EBP, C/EBPalpha, C/EBPbeta, C/EBPdelta, C/EBPepsilon, C/EBPgamma, C1, CAC-binding protein, CACCC-binding factor, Cactus, Cad, CAD1, CAF17, CAL, CAP, CAR2, CArG box-binding protein, CAT8, CAUP, CBF1, CBF2, CBF3, CBF4, CBF5, CBF-A, CBF-B, CBF-C, CBP, CBTF, CCAAT-binding factor, CCBF, CCF, CCG1, CCK-1a, CCK-1b, CCR4, CD28RC, CDC10, Cdc68, CDF, cdk2, CDP, CDP2, Cdx-1, Cdx-2, Cdx-3, Cdx4, CEBF CEF1, ceh-1, ceh-10, ceh-12, ceh-13, ceh-14, ceh-16, CEH-18 and (all ceh related factors), CeMyoD, c-Ets-1, C-Ets-1A, c-Ets-1B, CF1, Cfla, CF2-I, CF2-11, CF2-III, CFF, CG-1, CHA4, CHOP-10, Chox-2.7, Chx10, CIN5, CIIIB1, c-Jun, CKB3, Clox, c-Maf, CMB1, CMB2, c-Myb, c-Myc, CNBP, Cnc, CoMP1, core-binding factor, CoS, COUP, COUP-TF, CP1, CP1A, CP1B, CP1C, CP2, CPBP, CPC1, CPE binding protein CPRF-1, CPRF-2, CPRF-3, CPM10, CPM5, CPM7, CPPI, CPRF-1, CPRF-2, CPRF-3, CPRF4a, CPRF-4b, all CREB related factors, CRE-BP1, CRE-BP2, CRE-BP3, CRE-BPa, CreA, CREB, CREB-2, CREBomega, CREMalpha, CREMbeta, CREMdelta, CREMepsilon, CREMgamma, CREMtaualpha, CRF, all CRM related factors, Croc, Crx, CRZ1, CSBP-1, CtBP, CTCF, CTF, CUM1, CUM10, CUP2, CUP9, CUS1, Cut, Cux, CWH-1, CWH-2, CWH-3, Cx, cyclin A, cyclin T, cyclin T1, cyclin T2, cyclin T2a, cyclin T2b, CYS3, D-MEF, Da, all DAL related factors, DAP, DAP1, DAT1, DAX1, DB1, DBF-A, DBF4, DBP, DBSF, dCREB, DDB, DDB-1, DDB-2, dDP, dE2F, DEAP3, DEF, DEFH2, Delilah, delta factor, deltaCREB, deltaE1, deltaEF1, deltaMax, DENF, DENF1, DENF2, DENF3, DEP, DEP2, DEP3, DEP4, DERmo-1, DF-1, DF-2, DF-3, Dfd, dFRA, DHR3, DHR38, DHR78, DHR96, dioxin receptor, dJRA D1, DII, all Dlx related factors, DM-SSRP1, DMLP1, Dof3, DP-1, DP-2, Dpn, Drl, all DREB related factors, DRF1, DRF2, DRTF, DSC1, DSIF, DSP1, DST1, DSXF, DSXM, DTF, E, E1A, E2, E2BP, E2F, E2F-BF, E2F-I, E4, E47, E4BP4, E4F, E4TF2, E7, E74, E75, EAP1, EAP2L, EAP2S, EAR2, EBF, EBF1, EBNA, EBP, EBP40, EC, EC5, ECF, ECF2, ECF3, ECH, ECM22, EcR, eE-TP EF-1A, EF-C, EF1, EFgamma, EGM1, EGM2, EGM3, Egr, EGR2, EGR3, eH-TF, E1a, EivF, EKLF, Elf-1, Elg, Elk-1, ELP, Elt-2, ErBP-1, embryo DNA binding protein, Emc, EMP, EMF2, EMF3, EMF4, Ems, Ernx, Emx-1, Emx-2, En, ENH-binding protein, ENKTF-1, epsilonF1, ER, Erbeta, EREBP-1, EREBP-2, EREBP-3, EREBP4, ERF1, Erg, Esc, Escl, esg, Esx-1a, Esx-1b, ETF, ETL, Eve, Evi, Evx, Exd, Ey, en-1, en-2, f(alpha-f(epsilon), F27E5.2, F2F, FACB, F-ACT1, factor 1, factor 2, factor 3, factor B 1, factor B2, factor delta, factor I, FAR, Fbfl, FBF-A1, FBP, FBP1, FBP11, FBP2, FBP6, FBP7, f-EBP, FHL1, FIM, FKBP59, Fkh, FKH1, Fkh-1, FKH2, Fkh-2, Fkh-3, Fkh4, Fkh-5, Fkh-6, FKHR, FKHRL1, FKHRL1P1, FKHRL1P2, FKHRP1, FlbD, FLC, FLF, Flh, Fli-1, FLO, FLO8, FLV-1, FOG, FosB, FosB/SF, Fra-1, Fra-2, Freac-1, Freac-10, Freac-2, Freac-3, Freac4, Freac-5, Freac-6, Freac-7, Freac-8, Freac-9, FRG Y1, FRG Y2, EWF, FTS, Ftz, FIZ-F1, FTZ-Flbeta, FZF1G factor, G factor, G/HBF-1, G10BP, G6 factor, GA-BF, GABP, GABP alpha, GABP-beta1, GABP-beta2, GAF, GAF1, GAF2, GAG2, GAL11, GAL4, GAL80, GammaCAAT, gammaCAC1, gammaCAC2, gamma-factor, gammaOBP, GAMYB, GAT1, GAT2, GAT3, GAT4, GATA-1, GATA-LA, GATA-1B, GATA-2, GATA-3, GATA4, GATA-5, GATA-5A, GATA-, GATA-6, GATA-6A, GATA-6B, GBF, GBF1, GBF12, GBF1A, GBF1B, GBF2, GBF2A, GBF2B, GBF3, GBF4, GBF9, GBP, GC1, GC2, GC3, GCF, GCM, GCMa, GCMb, GCN4 GCN5, GCNF, GCR1, GCR2, GE1, GEBF-I, GF1, GFI, Gfi-1, GFII, GHF3, GHF-5, GHF-7, GIS1, GKLF, GL1, Gl15, G12, Glass, GLI, GLI3, GLN3, GLO, GM-PBP-1, GP, GR, GR alpha, GR beta, GRF-1, Grg-4, Grg-5, GRIP1, Groucho, Gsb, GSBF1, Gsbn, Gsc, Gsc A, Gsc B, Gt, GT-1, GT-2, GT-IC, GT-IIA, GT-IIBalpha, GT-IIBbeta, GTS1, Gtx, GZF3, H16, H1TF1, H1TF2, H₂B abp 1, H2RIIBP, H4TF-1, H4TF-2, HAC1, HAL9, HALF-I, HAP1, HAP2, HAP3, HAP4, HAP5, Hb, HB9, HBLF, HBP-1, HBP-1a, HBP-1a(1), HBP-1a(c14), HBP-1b, HIBP-1b(c1), HCM1, HDaxx, heat-induced factor, HEB, HEB1-p67, HEB1-p94, HEF-1B, HEF-1T, HEF4C, HEN1, HEN2, HeRunt-1, HES-1, HES-2, HES-3, HES-5, Hesxl, Hex, HFH-1, HFH-11A, HFH-11B, HFH-2, HFH-3, HFH-4, HFH-5, HFH-6, HFH-7, HPH-8, HIF-1, HIF-1alpha, HIF-1beta, HiNF-A, HiNF-B, HiNF-C, HiNF-D, HiNF-D3, HiNF-E, HiNF-M, HiNF-P, HIP1, HIR1, HIR2, H1R3, HIRA, HIV-EP2, HIlf, Hlf-alpha, Hlf-beta, HLX, Hlx, HMBP, HMG I, HMG I(Y), HiMG Y, HMGI-C, HMS1, HMS2, HNF-1, HNF-1A, HNF-1B, HNF-1C, HNF-3, HNF3(-like), HNF-3alpha, HNF-3B, HNF-3beta, HNF-3gamma, HNF-4, HNF4(D), HNF4alpha1, BNF4alpha2, HNF4alpha3, BNF-4alpha4, HNF4alpha7, HNF-4beta, HNF-4gamma, HNF-6, HNF-6alpha, HNF-6beta, hnRNP K, Hox11, HOXA1, HOXA10, HOXA10PL2, HOXA11, HOXA13, HOXA2, HOXA3, HOXA4, HOXA5, HOXA6, HOXA7, HOXA9, HOXB1, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC6 (PR1), HOXC6 (PR11), HOXC8, HOXC9, HOXD1, HOXD10, HOXD11, HOXD12, HOXD13, HOXD3, HOXD4, HOXD8, HOXD9, HP1 site factor, Hp55, Hp65, HrpF, HSE-binding protein, HSF, HSF1, HSF_, HSF24, HSF30, HSF8, hsp56, Hsp90, HST, HSTF, HY5, IBF, IBP-1, IBR, ICER, ICER-I, ICER-Igamma, ICER-II, ICER-Iigamma, ICP4, ICSBP, Id1, Id1.25, Id1H′, Id2 Id3, Id3/Heir-1, Id4, IDS1, IE1, IEBP1, IEFga, IF1, IF2, IFH1, IFNEX, IgPE-1, IgPE-2, IgPE-3, Ik-1, Ik-2, Ik-3, Ik4, Ik-5, Ik-6, Ik-7, Ik-8, IkappaB, IkappaB-alpha, IkappaB-beta, IkappaB-gamma IkappaB-gamma1, IkappaB-gamma2, IkappaBR, IK13, ILF, ILRF-A, IME1, IME4, IN02, IN04, INSAF, IPF1, I-POU, IRBP, IRE-ABP, IREBF-1, IRF-1, IRF-2, IRF-3, irIB-2a, Irx-3, ISGF-1, ISGF-3, ISGF-3alpha, ISGF-3gamma, Isl-1, ISRF, ISRFI, ITF, nT-1, ITF-2, IUF-1, Ixrl, JRF, Jun-D, JunB, JunD, K06B9.5, K07C11.1, kappaY factor, KAR4, KBF2, kBF-A, KBP-1, KCS1, KER1, -1, Kid-i, Kinl7, KN1, Kni, Knox3, KNRL, Koxl, Kr, Kreisler, KRF-1, Krox-20, Krox-24, Ku autoantigen, KUP, Lab, LAC9, LBP, LBP-1, LBP-1a, Lc, LCR-F1, LD, Ldbl, LEF-1, LEF-1B, LEF 1S, LEU3, LF-A1, LF-A2, LF-B2, LF-C, LFY, LG2, LH-2, Lhx-3, Lhx-3a, Lhx-3b, Lhx4, LHY, Lim-1, Lim-3, lin-1, lin-11, lin-14A, lin-14B1, lin-14B2, lin-29A, lin-29B, lin-31, lin-32, lin-39, LIP15, LIPl9, LIT-1, LKLF, Lmo1, Lmo2, Lmx-1, L-Myc1, L-Myc-1, L-Myc-1 (long form), L-Myc-1(short form), L-Myc-2, LR1, LSF, LSIRF-2, LUN, Lva, LVb-binding factor, LVc, LXRalpha LyF-1, Lyl-1, LYS14, Lz, M factor, M-Twist, M1, m3, Mab-18, MAC1, Mad, MAF, MafB, MafF, MafG, MafK, Mal63, MAPF1, MAPF2, MASH-1, MASH-2, mat-Mc, mat-Pc, MATal, MATalphal, MATalpha2, MATH-1, MATH-2, Max1, M factor, M1, m3, Mab-18 (284 AA), Mab-18 (296 AA), mab-5, MAC1, Madl, Mad3, Mad4, MADS1, MADS11, MADS16, MADS2, MADS24, MADS3, MADS4, MADS45, MADS5, MADS6, MADS7, MADS8, MADS9, MAF, MafB, MafF, MafG, MafK, MAL13, MAL23, MAL33, MAL63, MAPF1, MAPF2, MASH-1, MASH-2, Matl-Mc, MATal, MATalphal, MATalpha2, MATH-1, MATH-2, mat-Pc, Max, Max1, Max2, MAZ, MAZ1, MB67, MBF1, MBF-1, MBF2, MBF3, MBF-I, MBP1, MBP-1 (1), MBP-1 (2) MBP-2, MCBF, MCM1, MCM1+MATalpha1, MDBP, MDBP-2, MDS3, mec-3, MECA, MED11, MBD2, MED4, MED6, MED7, MED8, mediating factor, MEF1, MEF-2, MEF-2B, MEF-2B-1, MEF-2B-2, MEF-2B-3, MEF-2B4, MEF-2C, MEF-2C (433 AA form), MEF-2C (465 AA form), MEF-2C (473 AA form), MEF-2C/delta32 (441 AA form), MEF-2D, M-2D (506 AA form), MEF-2D (514 AA form), MEF-2D00, MEF-2DOB, MEF-2DA-0, MEF-2DA-B, MEF-2DA0, MEF 2DAB, Meis-1, Meis-1-1, Meis-1-2, Meis-1-3, Meis-1-4, Meis-1a, Meis-1b, Meis-2a, Meis-2b, Meis-2c, Meis-2d, Meis-3, Mesol, MET18, MET28, MET31, MET32, MET4, Mf2, MF3, MFH-1, Mfh-1, MGA1, Mhox, MHR1, M1, MIP1, MIF-1, MIG1, MIG2, Mix.1, Mix.2, Mix.3, Mix.4, Mixer, MIITA, Miz-1, MKR2, MLP, MM-1, MNBia, MNBlb, MNF1, MNR2, MOK-2, MOP3, MOT1, MOT3, MP4, MPBF, MR, MRF4, MRR, Msh, MSN1, MSN2, MSN4, Msx-1, Msx-2, MIB Zf, MTF1, MTF-1, MTH1, Mt11, mtTF1, M-Twist, muEBP-B, muEBP-C2, MUF1, MUF2, Mxi1, MYB A, MYB.PH1, MYB.PH2, MYB.PH3, MYB 1, Myb-1, all Myb related proteins, MYB-P1, MYBST1, myc-CF1, myc-PRF, MYC-RP, Myef-2, Myf-3, Myf-4, Myf-5, Myf-6, Myn, MyoD, Myogenin, MZF-1, Nabl, Nau, NBF, NC1, NCB2, NDT80, NELF, NeP1, NER1, Net, NeuroD, NF III-a, NF III-c, NF III-e, NF-1, NF-1/L, NF-1/Redl, NF-1A, NF-1A1, NF-1A1.1, NF-1A2, NF-1A3, NF-1A4, NF-1A5, NF-1B, NF-1B1, NF-1B2, NF-1B3, NF-1B4, NF-1C1, NF-1C2, NF-1C4, NF-1X NF-1X1, NF-1×2, NF-1×3, NF2d9, NF-4FA, NF-4FB, NP4FC, NF-A, NF-A3, NF-AB, NFalpha1, NFalpha2, NFalpha3, NFalpha4, NF-AT, NFAT-1, NF-AT3, NF-Atc, NF-ATc3, NF-Atp, NF-Atx, NF-BA1, NfbetaA, NF-CLEOa, NF-CLEOb, NF-D, NFdeltaE3A, NFdeltaE3B, NFdeltaE3C, NFdeltaE4A, NFdeltaE4B, NFdeltaE4C, Nfe, NF-E, NF-E1b, NF-E2, NP-E2 p45, NF-E3, NF-E4, NFE-6, NF-EM5, NF-Gma, NF-GMb, NF-H1, NF-H2, NF-H3, NFH3-1, NFH3-2, NF13-3, NPH3-4, NF-IL-2A, NP-IL-2B, NF-InsE1, NF-InsE2, NF-InsE3, NF-jun, NF-kappaB, NF-kappaB(-like), NF-kappaB 1, NF-kappaB I precursor, NF-kappaB2, NF-kappaB2 (p49), NF-kappaB2 precursor, NF kappaE1, NF-kappaE2, NF-kappaE3, NF-lambda2, NF-MHCIIA, NF-MHCIIB, NF-muE1, NP-muE2, NF-muE3, NF-muNR, NF-ODC1, NF-S, NP-TNF, NF-U1, NP-W1, NF-W2, NF-X, NF-X1, NF-X2NF-X3, NF-Xc, NF-Y, NF-Y′, NF-YA, NP-YB, NF-YC, NF-Zc, NF-Zz, NGFI-B, NGFI-C, NHP-1, NHP-2NHP3, NHP4, NHR1, NIP, NIRA, NIT2, NIT4, Nkx-2.1, Nkx-2.2, Nkx-2.5, NLS1, NMH7, NMHC5, Nmi, N-Myc, N-Mycl, N-Myc2, nob-1A, nob-1B, N-Oct-2alpha, N-Oct-2beta, N Oct-3, N-Oct-4, N-Oct-5a, N-Oct-5b, NOR1, NOT, NOT1, NOT2, NOT3, NOT5, NP-rn, NP-IV, NP-TCII, NP-Va, NPX1, NRD I, Nrf1, NRF-1, Nrf2, NRF-2NRF-2beta1, NRF-2gamma1, NRFA, NRG1, NRG2, NRL, NS-1, NSDD, NTF, NTF1, NUC-1, Nur77, NUT1, NUT2, OBF, OBF-1, OBF3.1, OBF3.2, OBF4, OBF5, OBP, OBPi, OC-2, OCA-B, OCSBF-1, OCSTF, Oct-1, Oct-10, Oct-11, Oct-1A, Oct-1B, Oct-1C, Oct-2, Oct-2.1, Oct-2.3, Oct-2.4, Oct-2.6, Oct-2.7, Oct-2.8, Oct-2B, Oct-2C, Oct4, Oct-4A, Oct-4B, Oct-5, Oct-6, Oct-7, Oct-8, Oct-9, Octa-factor, octamer-binding factor, oct-B2, oct-B3, Oct-R, Odd, ODR7, OG-12, OG-2, OG-9, OHP1, OHP2, Olf-1, OM1, ONR1, Opaque-2, OPM1, OSBZ8, Otd, Otx1, Otx2, Otx4, Ovo, OZF, P (long form), P (short form), P1, p107, p130, p28 modulator, p300, p38erg, p40x, p45, p49erg, p53 as, p55, p55erg, p58, p65delta p67, PAB1, PacC, PAF1, pag-3, PAGL1, pal-1, Pap1+, par-2, Paraxis, PARP, Pax-1, Pax-1/9, Pax-1/9 (AmphiPax-1), Pax-1/9-I, Pax-1/9-II, Pax-1/9-111, Pax-1/9-IV, Pax-1/9-V, Pax-1/9-VI, Pax-2, Pax-2.1, Pax-2.2, Pax-2/5/8, Pax-2a, Pax-2b, Pax-3, Pax-3A, Pax-3B, Pax-4, Pax-4a, Pax-4b, Pax-4c, Pax-4d, Pax-5, Pax-6, Pax-6 (Pax-QNR), Pax-6/Pd-5a, Pax-6 12.1, Pax-6 12.2, Pax-6 4.1, Pax-6 4.2, Pax-6 J2, Pax-7, Pax-8, Pax-8a, Pax-8b, Pax-8c, Pax-8d, Pax-8e, Pax-8f, Pax-8g, Pax-9, Pax-A, Pax-B, Pb, PBF, PBP, Pbx-1a, Pbx-1b, Pc, PC2, PC4, PC4 p9, PC5, Pcrl, PCRE1, PCT1, PDM-1, PDM-2, PDR1, PDR3, Pdx-1, PEA1, PEA2, PEA3, PEB1, PEBP2, PEBP2alpha, PEBP2alphaA/Osf2, PEBP2alphaA/til-1, PEBP2alphaA/til-1 (Y), PEBP2alphaA/til-1(U), PEBP2alphaA1, PEBP2alphaA2, PEBP2alphaB1, PEBP2alphaB2, PEBP2beta, PEBP2beta1, PEBP2beta2, PEBP2beta3, PEBP5, Pep-1, PERIANTIA, pes-lapes-1b, PF1, PF3, PGA4, PGD1, pha4, PHAN, PHD1, phiAP3, PHO2, PHO4, PHO80, Phox-2, php-3, P1, P11, P12, pie-1, PIHbox9, PIP2, Pit-1, Pit-1a, Pit-1b, Pit-1c, Pitx-3, PLE, PLE1/DEFH200, PLE/DEFH49, PLE/DEFH72, PLE/SQUA, PLZF, PNPI2, PO-B, pointedP1, pointedP2, Pontin52, pop-1POP2, POTM1-1, pou[c], Pou2, pox neuro, PP1, PP2, PPAR, PPARalpha, PPARbeta, PPARgamma, PPR1, PPUR, PPYR, PR PR A, PRb, Prd, PRDI-BF1, PRDI-BFc, PREB, Prop-1, protein a, protein b, protein c, protein d, PRP, PSE1, Psx-1, Psx-2, P-TEFb, PTF, PThL, PTF1-alpha, PTF1-beta, PTFalpha, PTFbeta, PTFdelta, PTFgamma, Ptx-1, Ptx-2, Ptx-2B, Pu box binding factor, Pu box binding factor (BJA-B), PU.1, Pu.1, PUB 1, PuF, PUF-I, Pur factor, Pur-1, PUT3, P-wr, PX, PZF1, qa-1F, QBP, QUT1, R, R1, R2, RAD1, Rad-1, RAD18, RAD2, RAF, RAP1, RAP2.5, RAR, RAR-alpha, RAR-alpha1, RAR-alpha2, RAR-beta, RAR-beta1, RAR-beta2, RAR-beta3, RAR-beta4, RAR-gamma, RAR-gamma1, RAR-gamma2, RAV1, RAV2, Rax, Rb, RBP60, RBP-Jkappa, Rc, RC1, RC2, RCS1, REB, REB1, Reb1p, Re1A, Re1B, repressor of CAR1 expression, REV-ErbAalpha, REX-1, RF1, RF2a, RFX, RFX1, RFX2, RFX3, RFX5, RF-Y, RGM1, RGR1, RGT1, RIC1, RIM1, RIP14, RITA 1, RLM1, RME1, RMS1, Ro, Roaz, ROM1, ROM2, RORalpha1, RORalpha2, RORalpha3, RORbeta, RORgamma, Rox, Roxl, ROX3, RPF1, RPGalpha, RPH1, RREB-1, RRF1, RRF2, RRF3 RRN10, RRN11, RRN3, RRN5, RRN6, RRN7, RRN9, RS2, RSC4, RSRFC4, RSRFC9, RSV-EF-11 RTF1, RTG1, RTG2, RTG3, Runt, RVF, Rx, Rx1, Rx2, Rx3, RXR-alpha, RXR-beta, RXR-beta1, RXR-beta2, RXR-gamma, S8, SAP1, SAP-1a, SAP-1b, SBF, SBF-1, Sc, SCBPalpha, SCBPbeta, SCBPgamma, SCD1/BP, SCM-inducible factor, Scr, S-CREM, S-CREMbeta, Sd, Sdc-1, SDS3, SEF1, SEF-1 (1), SEF-1 (2), SEF3, SEF4, SEM-4, SET1, SET2, SF1, SF-1, SF-2, SF-3, SF-A, SFL1, SGC1, SGF-1, SGF-2, SGF-3, SGF-4, Shn, SHP, SHP1, SHP2, SIF, SIG1, Sm, Sm-pllo, SfI-p15 SfI-p18, Sim1, Sim2, Six-1, Six-2, Six-3, Six-3alpha, Six-3beta, Six4, Six-4A, Six-4B, Six-4C, Six-5, Six-6, Skn-1, SKN7, SKO1, SLM1, SLM2, SLM3, SLM4, SLM5, Slp1, slp2, S-Myc, Sn, SN (sienna), Sna, SNF5, SNF6, SNP1, So, SOX-11, SOX-12, Sox-13, SOX-15, Sox-18, Sox-2, Sox-4, Sox-5, SOX-6, SOX-9, Sox-LZ, Sp1, Sp2, Sp3, Sp4, SPA, spE2F, Sph factor, Sp1-B, SpOtx, Sprin-1, SpRunt-1, SQUA, SRB10, SRBil, SRB2, SRB4, SRB5, SRB6, SRB7, SRB8, SRB9, SRD1, SR1 BP, SREBP-1, SREBP-1a, SREBP-1b, SREBP-1c, SREBP-2, SREP, SRE-ZBP, SRF, SRY, Sry h-1 Sry-beta, Sry-delta, ssDBP-1, ssDBP-2, SSRP1, Staf, Staf-50, STAT, STAT1, STATlalpha, STATibeta, STAT2, STAT3, STAT4, STAT5, STAT5A, STAT5B, STAT6, STC, STD1, Ste11, STE12, STE4, STF1, STF2, STKA, STM, STP1, Stral3, StuAp, su(f), Su(H), su(Hw), SUM-1, SUP, SVP, SVP46, SVI/SNF complex, SWIL, SWI2, SWI3, SWI4, SWI5, SWI6, SWP, T-Ag, t-Pou2, T3R, T3R-alpha, T3R-alpha1, T3R-alpha2, T3R-beta, T3R-beta1, T3R-beta2, TAB, T-Ag, TAG1, Tal-1, Tal-1beta, Tal-2, TAR factorTat, Tax, TCF, TCF-1TCF-1A, TCF-1B, TCF-1C, TCF-1D, TCF-1E, -1F, TCF-1G, TCF-2, TCF-2alpha, TCF-3, TCF-3B, TCF-3C, TCF-3D, TCF4, TCF-4(K), TCF-4B, TCF-4E, TCF-A, TCF-B, TCFbetal, TDEF, TEA1, TEC1, TEF, TEF 1, TEF-1, TEF2, TEF-2, Te1, TF68, TFE3, TFE3-L, TFp3-S, TFEB, TFEC, TFIIA, TFIIA (13.5 kDa subunit), Tf-LF1, Tf-LF2, TF-Vbeta, TGA, TGA1, TGAla, TGA2, TGA3, TGA6, TgF1, TGGCA-binding protein, TGT3, Th1, THM1, THM18, THM27, THRA1, TIF1, TIF2, TIN-1, TINY, TIP, tI-POU, TLE1, T11, Tlx, TM3, TM4, TM5, TM6, TM8, TMF, t-Pou2, TR2, TR2-11, TR2-9, TR3, TR4, Tra-1 (long form), Tra-1 (short form), TRAP, TREB-1, TREB-2, TREB-3, TREF1, TREF2, TRF, TRF (2) Trident, TSAP, TSF3, Tsh, TIF-1, TTF-2, TTG1, Ttk 69K, Ttk 88K, TTP, Ttx, ttx-3, TUBF, Twi, TXREF, TyBF, UAY, UBF, UBF1, UBF2, UBP-1, Ubx, UCRB, UCRF-L, UEF-1, UEF-2, UEF-3, UEF4, UF1-H3beta, UFA, UFB, UFO, UGA3, UHF-1, UME6, unc-30, unc-37, unc4, Unc-86, URF, URSF, URTF, USF, USF2, vab-3, vab-7, vaccinia virus DNA-binding protein, Vav, Vax-1, Vax-2, VBP, VDR, v-ErbA, VETF, v-Ets, v-Fos, vHNF-1, vHNF-1A, vF-1B, vHNF-1C, VITF, v-Jun, v-Maf, Vmw65, v-Myb, v-Myb/v-Ets, V-Myc, v-Myc, Vpl, Vpr, v-Qin, v-Re1, VSF-1, WC1, WC2, Whn, WT1, WT1I, WZF1, X-box binding protein, X-Twist, X2BP, xaml, X-box binding protein, XBP-1, XBP-2, XBP-3, XF1, XF2, XFD-1, XFD-2, XFD-3, XFG20, XGRAF, Xiro1, Xiro2, Xiro3, xMEF-2, XPF-1, XrpFI, XW, XX, yan, YB-1, YB-3, Ybx-3, YEB3, YEBP, Y1, YNG2, YPF1, YY1, ZAP, ZEB, ZEML, ZEM2/3, Zen-1, Zen-2, Zeste, ZF1, ZF2, ZF5, Zfh-1, Zfh-2 Zfp-35, ZID, ZIP-1A, ZIP-2A, ZIP-2B, ZM1, ZM38, Zmhoxla, Zn-15, ZNF174, ZPT2-1, ZPT2-2, ZPT2-3, ZPT2-4, Zta. In addition, any factors which retain the ability to regulate gene expression, either through activation or repression, and are as of yet previously undiscovered or uncharacterized are covered by the present invention.

5.6 Basic Biology Applications of Transcription Factor Target Gene Microarrays

The study of gene regulation as it relates to cellular and even organismal biology is essential for the thorough understanding of events which occur at the molecular level to initiate and maintain biological processes which drive embryonic development or ensure survival. By assessing the activation and/or repression of genetic loci known or predicted to plays roles in particular aspects of physiology, it is possible to correlate transcriptional regulatory mechanisms with specific phenotypes.

Nucleotide microarrays of transcription factor targets allow for the narrowed and focused assessment of expression profiles of genes relevant to the hypotheses being addressed. Directing attention only to genes which are known or thought to play roles in a particular facet of biology saves much time and expense as needless irrelevant expression profiles are not pursued. For example, the study of cell cycle control and cell division is at the forefront of cancer research and promises to ultimately provide avenues for treatment of this devastating disease. A great deal of these studies focus on cell lines which progress temporally from a nontumorigenic state to a cancerous phenotype. Microarray analysis of targets for transcription factors such as the tumor suppressor p53 (E1-Diery et al., 1993, Cell, 75: 817-825) and Rb (Iunaief et al., 1994, Cell, 79(1):119-30) utilizing these lines as RNA sources will undoubtedly reveal distinct genetic profiles for each stage of tumor progression. A unique transcriptional profile or “transcriptome” may be obtained at different temporal points during progression of the tumorigenic phenotype. Information gleaned from target nucleotide microarray studies of this nature not only provides unique fingerprints of cellular physiology but also reveals potential mechanisms that drive deviation from the normal cellular fate.

5.7 Medical Applications of Transcription Factor Target Gene Microarrays

The presently described invention entails the creation of physiologically focused arrays and microarrays through the annotation and organization on solid phase supports of transcription factor target genes. Transcription factors may be chosen which represent a particular clinical aspect of physiology, based upon previous research implicating these factors in said areas and the targets for these factors efficiently identified and arrayed. The inherent ability of these factors to home in on target genes through either their DNA binding domains or through interactions with other proteins is exploited utilizing previously described technology (PCT patent application serial number PCT/US01/24823, filed Aug. 14, 2000 and herein incorporated by reference).

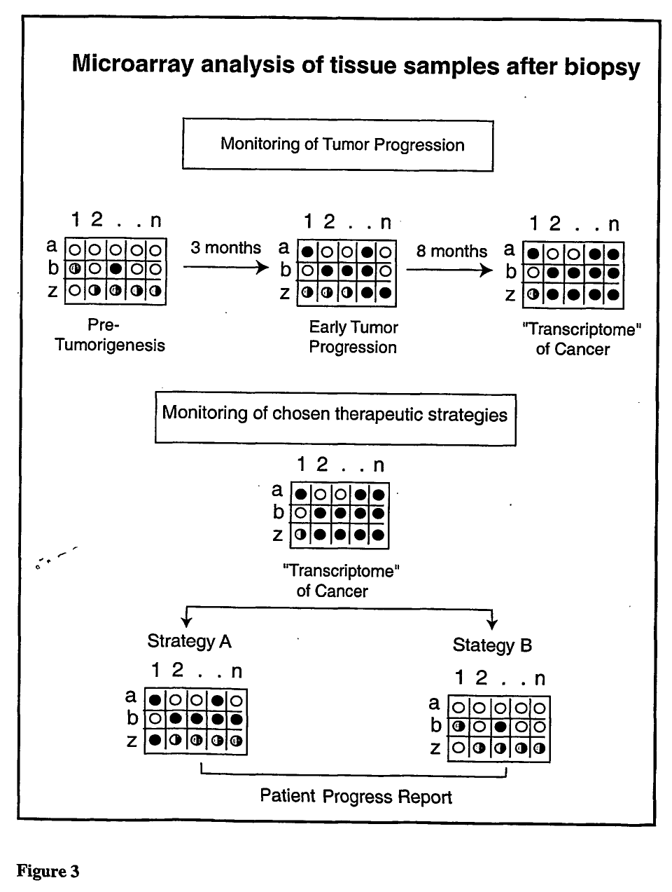

FIG. 3 is an illustrative example of the expressional characterization of a series of biopsied human tissue samples as disease progresses from no overt morphological alterations to a cancerous phenotype. An expression profile of transcription factor target genes is taken at different temporal stages. Therapeutic strategies may be implemented and subsequent microarray expression profiles analyzed to monitor the effectiveness of the therapy. A reversion to profiles similar to those for early tumor progression or pre-tumorigenesis suggests effective treatment. In the theoretical example of FIG. 3 therapeutic strategy B reverts tumor progression to near pretumorigenic stages. It is contemplated and therefore covered by the present invention that virtually any type of cancer may be effectively monitored by transcription factor target nucleotide arrays and microarrays. It is also contemplated and therefore covered by the present invention that maladies other that those related to a cancerous phenotype may also be monitored via transcription factor target nucleotide microarrays. These include, but are in no way limited to inherited as well as sporadic conditions. As mentioned above, not only are revealing expression profiles discerned from such transcription factor target nucleotide microarrays but potential points of therapeutic intervention or even therapeutic target discovery may be uncovered. In addition, patient prognosis may be significantly improved with “standardized” expression profiles of transcription factor targets for both normal and diseased tissue at different stages of progression. Finally, and perhaps the most intriguing aspect of the technology, is the ability to diagnose a disorder based upon gene expression patterns prior to the establishment of any overt symptoms. Table 1 illustrates this point by providing a “transcriptome” of p53 targets for a number of tissue samples known to be isolated at different stages of cancer progression (T1 through T8). Note that a unique expression level is annotated for each target gene a any given timepoint during tumor progression. In addition, other stimuli such as environmental cue., and age may be correlated with a categorization of gene expression profiles. Table 2 illustrates a compendium of alterations in target gene expression based upon internal and external influences. Data accumulated from these profiles can undoubtedly yield significant insight into diagnostic applications as well as the development of preventative strategies.

5.8 Transcription Factor Target Protein Arrays

The presently described invention details the construction and implementation of transcription factor target protein arrays and microarrays for the purposes of identifying interactions of these target proteins with chemical molecules, nucleotide sequences and other proteins of enzymatic or nonenzymatic origin.

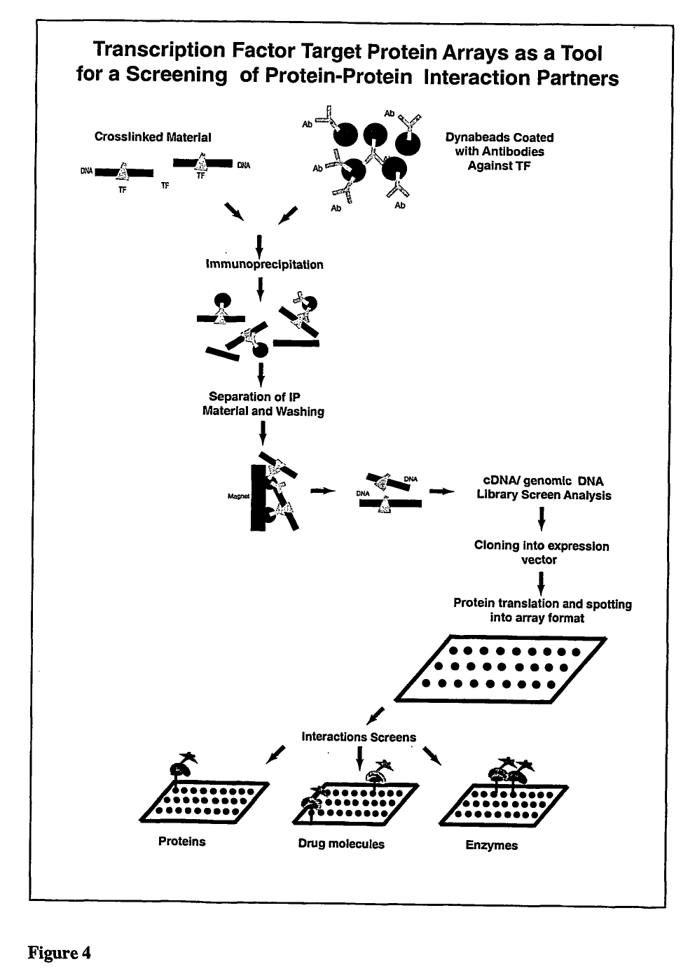

FIG. 4 illustrates the scope of the process from the identification of transcription factor target genes to the characterization of target protein interacting molecules through the utilization of nonliving target protein arrays. As described previously, transcription factor/DNA complexes are cross-linked in vivo via the addition of formaldehyde to cells in tissue culture or to isolated living tissues themselves. In the presently described invention, antibody coated Dynabeads™ (Dynal Corporation) are added directly to cross-linked material and specific antibody/transcription factor/target gene complexes are immunoprecipitated, washed and DNA fragments representing target genes of interest subsequently isolated (PCT patent application serial number PCT/US01/24823, filed Aug. 14, 2000 and herein incorporated by reference). Pools of these fragments contain genomic sequences corresponding to actively transcribed regions of transcription factor target loci. These sequences are screened against appropriate cDNA expression libraries to quickly and efficiently purify transcription factor target genes in the context of expression vectors for rapid production of the corresponding proteins. cDNAs corresponding to transcription factor target genes are translated and transcription factor target protein products are subsequently arrayed into a format suitable for interaction screening. These arrays are typically of a “living” or “nonliving” nature (see below). Screens are implemented for the discovery of specific interactions between transcription factor target proteins and other proteins/enzymes, nucleotide sequences, metals or small molecule drugs. It is contemplated and therefore covered by the present invention that any molecule identifies through the utilization of transcription factor target protein arrays may represent a potential avenue of therapy for particular aspects of human physiology and disease. Interactions may be identified b) a number of methods but most often are revealed via fluorescent tags conjugated to the screen candidates of interest (MacBeath et al., 2000, Science, 289: 1760-1763). Other biochemical detection methods include, but are in no way limited to radioactive hybridization, colorimetric detection and enzymatic activity such as that of horse radish peroxidase (HRP). It should be noted that full-length transcription factor target protein sequences are not necessarily needed to produce interaction results, but rather target peptide or short amino acid sequences alone may be sufficient.

5.9 Advantages of the Presently Described Invention over Existing Technology

While it is evident that nucleotide array and microarray characterization of gene expression patterns within specific sample populations is now a reality, a number of conceptual problems exist which must be overcome for the technology to become routine. Reproducible data output is a primary concern. The amount of sequence redundancy in the human genome is considerable. The presently described invention aims to overcome this limitation by narrowing the scope of genes analyzed to only a subset of those contained in the genome. A more limited and physiologically focused number of genes per microarray decreases redundancy and cross-hybridization issues and results in more accurate expression profiling. As well, specificity in analysis will increase based upon smaller physiologically directed arrays. More specific analyses means more comprehensive data accumulation during each round of expression characterization, thus eliminating errors introduced by large-scale characterization of irrelevant loci.

In addition, current microarrays lack the utility of control loci needed to ensure correct administration of experimental procedures. The presently described invention eliminates this issue by providing numerous previously published known transcription factor target genes as controls for each physiologic and/or disease oriented nucleotide microarray. These controls are not only expressed in the appropriate temporal and spatial manner, but often play functional roles related to the physiology being characterized. For example, an array of target genes for the tumor suppressing transcription factor p53 (see FIG. 1) will contain a number of known targets, such as the WAF1 locus, which are shown to have functionality in regulating cell cycle and have affected expression patterns in tumorigenic tissue samples (E1-Diery et al., 1993, Cell, 75: 817-825). The appropriate controlling of nucleotide microarray analysis will consistently reveal reproducible experimental results.

Sample availability also poses a unique problem to the microarray analysis of gene expression profiles. The majority of DNA microarray experiments utilize sample RNA which has been harvested from at least 10⁶-10⁷ cells. In some cases, especially those involving human tissue, sample size is small and therefore rate limiting. Minute sample sizes may limit the number of microarray studies which may be performed as the larger the array of genetic loci the more sample required to get accurate readout and data acquisition. This is especially true given the enormous complexity of the genes present within currently existing arrays and microarrays, most of which are likely to be irrelevant to the particular sample or aspect of physiology being studied. By directing characterization of samples to microarrays which are focused on particular aspects of physiology an, disease, these arrays allow for the characterization of expression profiles for very limited sample sizes and increase the number of focused expression profiling experiments which may be undertaken. In addition, given the large number of sequences which are annotated and linked to support material, the cost of construction of nonfocused nucleotide arrays and microarrays is quite significant. The presently described invention circumvents this problem by focusing array construction and utilization only upon specific genes which play roles in particular aspects of physiology and disease.

While much progress has been made with respect to the high-throughput identification of biochemical interactions in both a biological and chemical context through the construction and use of protein arrays, several potential drawbacks of this technology limit its utility in the larger scope of optimizing the efficiency of biochemical interaction characterization and ultimately drug development (MacBeath et al., 2000, Science, 289: 1760-1763 and for review see Emili et al., 2000 Nature Biotechnology, 18: 393-397). Perhaps the most relevant limitation of the above described methodologies is the shear magnitude of labor required to construct the arrays, either of a living or nonliving origin. Given the estimated number of genes present in the human genome (at present 26,000) it would be extremely labor intensive and costly to organize the protein products of all such loci into biochemical arrays (Venter et al., 2001, Science, 291: 1304-1351). In order to gain the maximum value and utility of protein arrays it is necessary to strategically choose the proteins which are to be organized and annotated in the array format. Properly choosing which proteins or peptide sequences are to be included in each particular array will result in a focus on specific realms of physiology and even human disease, increasing the possibility of studying the appropriate interacting partners and ultimately developing therapeutics for the treatment of disease. As transcription factors typically have been demonstrated time and again to control certain specific aspects of cellular and developmental biology, it is evident that the inherent ability of these factors to dictate discrete gene expression patterns allows for an excellent opportunity to define which gene products (proteins) mar be included in each array. By organizing specific transcription factor target proteins into arrays the biochemical nature of entire realms of physiology and even disease can be studied thoroughly and efficiently.

### 6.0 EXAMPLES

6.1 Construction and Utilization of Transcription Factor Target Nucleotide Microarrays

FIG. 2 is a flowchart representation of transcription factor target glass chip microarray construction. Modified sequential chromosomal immunoprecipitation is performed on sonicated cross-linked chromatin isolated from cell lines and/or tissues (PCT patent application serial number PCT/US01/24823, filed Aug. 14, 2000 and herein incorporated by reference). Upon reversal of cross-linkage precipitated DNA fragments containing putative transcription factor target genes are screened either via I-PCR or against cDNA libraries. I-PCR results in the identification of promoter and enhancer elements specific for the transcription factor being studied and confirmation of direct target identity. cDNA library screening reveals valuable 5′ untranslated and coding sequence information crucial to expression pattern characterizations. Sequences are organized in a two-dimensional grid format for ease of target gene identification and analysis.

FIG. 3 illustrates the use of transcription factor target nucleotide microarrays for monitoring cancer patient prognosis during and prior to therapy. Each square within the grid contains specific oligonucleotide sequences corresponding to p53 target genes and linked covalently to the solid support. As RNA isolated from samples (or corresponding cDNA) is passed over the chip, evidence of target gene expression and quantitative analysis of levels is revealed by a change in light illumination for particular and specific target loci. A temporal change in expression patterns is indicative of transcriptome alteration during tumor progression. In addition, therapeutic effectiveness may be monitored through expression profiling as evidenced by changes in gene expression. A reversion of patient transcriptome outputs to that of early tumor progression or even pretumorigenesis is indicative of effective therapeutic strategies. In the illustrative example of FIG. 3 therapeutic strategy B reverts sample expression profiles to a pretumorigenic phenotype.

Table 1 is an example of temporal changes in gene expression patterns and levels as progression occurs from the normal to the tumorigenic phenotype. Samples T1 through T7 represen controls for different known temporal stages of tumorigenesis (from early to late) while samples N1 through N3 are unknown samples. Numbzrs listed linearly correlate with gene expression levels. Note how certain transcription factor targets are activated while others are repressed upon phenotype manifestation. From the data collected it is apparent that sample N1 correlates with an earlier manifestation of the disease as expression profiles are similar to that for sample T2. N2 exhibits a late stage expression profile resembling that of T5 and N3 shows no correlation to the disease phenotype.

Table 2 is a similar example of transcription factor target gene microarray expression classification upon issuance of external as well as internal influences. These influences in this particular example include various environmental stimuli such as exposure to carcinogens as well as age. Note how the expression profile of samples from patient A correlate with those in standard sample 1 while patient B samples exhibit similar expression profile to standard sample 3.

6.2 Transcription Factor Target Protein Nonliving Arrays

The ability to detect specific interactions of nucleotide sequences, small molecules, enzymes and other proteins with transcription factor target proteins allows for the ultimate design of therapeutics with higher efficacy and fewer side effects than those currently available. Several different types of applications of transcription factor target microarray proteomics can be employed to achieve the desired results. As mentioned above, there are primarily two types of array and microarray protein interaction screens which have been successfully utilized for the purposes of high-throughput interaction characterization (for review see Emili et al., 2000, Nature Biotechnology, 18: 393-397). The presently described invention optimizes and focuses each of these methodologies for the analysis and characterization of various entities which may interact with transcription factor target proteins. FIG. 5 is a diagrammatic flowchart of methodology employed for the utilization of “nonliving”/chemical transcription factor target protein microarrays. Peptide sequences or bacterially expressed glutathione-S-transferase fusion proteins are immobilized on a solid phase support such as a nylon membrane or glass chip in a hydrated, folded state to preserve the naturally occurring 3-dimensional structure of the protein (Martzen et al., 1999, Science, 286: 1153-1155). A number of assays may subsequently be implemented to determine the possibility of enzyme/substrate as well as simple protein/protein and protein/small molecule interactions. Transcription factor target proteins present in the array may be tested as targets for enzymatic action by observing modification of the arrayed proteins upon incubation with the enzyme of interest. Modifications such as phosphorylation or acetylation provide convenient tags which can be readily identified in vitro. In addition, it is possible to characterize the interactions of transcription factor target proteins with those present in virtually any type of cell through the passage of whole cell lysates over the arrays. Extensive washing and elution of bound proteins followed by mass spectrometry greatly enhances the scope of arrayed transcription factor target protein/protein interaction studies (Gygi et al., 1999, Nature Biotechnology, 17: 994-999; Neubauer et al., 1997, Proc. Natl. Acad. Sci. USA, 94: 385-390 and Lamond et al., 1997, Trends Cell Biol., 7: 139-142).

Finally, phage display methodologies complement transcription factor target protein array technology by allowing for the characterization of amplified libraries of proteins from virtually any source (Zozulya et al., 1999, Nature Biotechnol., 17: 1193-1198 and Hufton et al., 1999, J. Immunol. Methods, 231: 39-51). Bacteriophage samples expressing proteins on the surface of the phage are passed in contact with the transcription factor target protein array (FIG. 5). Only specific interactions between the arrayed target proteins and those on the outer shell of the bacteriophage will allow for binding of the phage to specific targets within the array after rigorous washing. These phage can be subsequently eluted from the protein array and the cDNA corresponding to the surface protein of interest can be purified and sequenced to reveal the protein's genetic identity and amino acid composition.

6.3 Transcription Factor Target Protein Living Arrays

As mentioned above, it is also possible to construct biological/“living” arrays of transcription factor target proteins as described in yeast (Uetz et al., 2000, Nature, 403: 623-627). The use of these transcription factor target protein arrays is illustrated diagrammatically in FIG. 6. Arrays of yeast colonies containing protein open reading frameIGAL4 activation domain fusions are mated with strains of yeast containing a single GAMA DNA binding domain fusion. Upon nutritiona selection only yeast clones in which interaction between the two GAJA fusion proteins occurs will survive due to recruitment of the activation complex to a nutritional supplement/GAL4 DNA binding site locus engineered within the yeast genome. Although GAJA interaction methodologies are described in the present invention, it is in no way limited to this particular transcription factor interaction and activation capacity. Other transcription factors and their prospective binding sites may be utilized for the successful detection of protein/protein interactions and are therefore covered by the present invention. Preparations of purified nucleotide sequences containing the cDNA encoding the interacting partner of interest are then performed from surviving yeast colonies. DNA sequencing of these fragments will reveal the identity of the array tag containing the interaction partner.

The retrieval of information on protein/protein, protein/small molecule and enzyme/substrate interactions for transcription factor target proteins is of considerable value for the development of therapeutic agents. Yet these data must be organized in a fashion that maximizes value and minimizes the complexity of the information at hand. The presently described invention therefore describes the importing and organization of all data corresponding to the interactions of transcriptior factor targets into proteomics interaction databases which are easily searchable for the desired biochemical interaction information (FIGS. 5 and 6). By implementing a vigorous bioinformatics platform to annotate and categorize these data researchers will have the opportunity to rapidly identify relevant transcription factor target protein interaction information for the ultimate design of therapeutics. This type of annotation will speed the identification and exploitation of therapeutically relevant transcription factor target proteins.

6.4 Therapeutic Discovery Utilizing Transcription Factor Target Protein Arrays

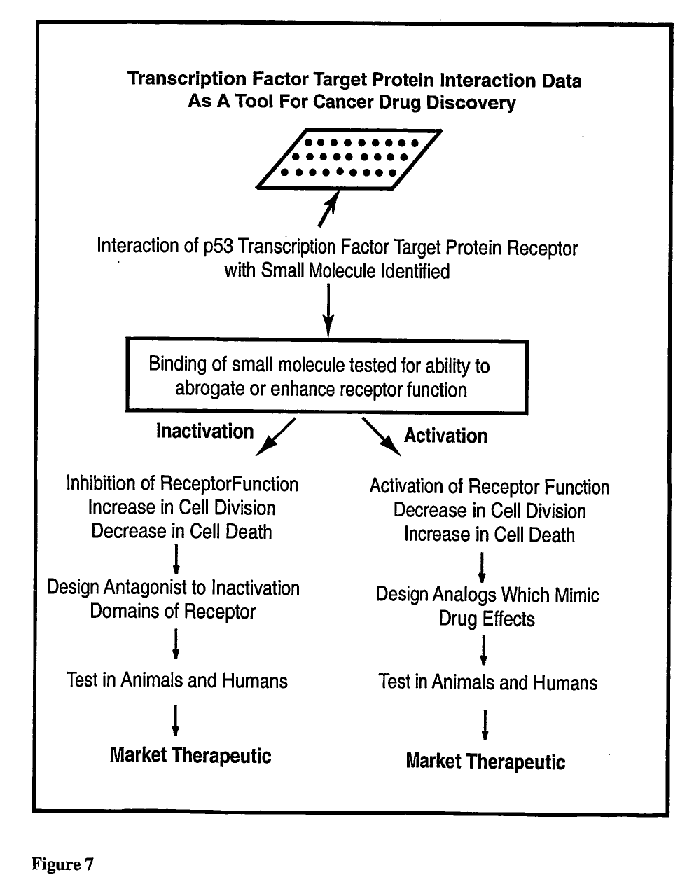

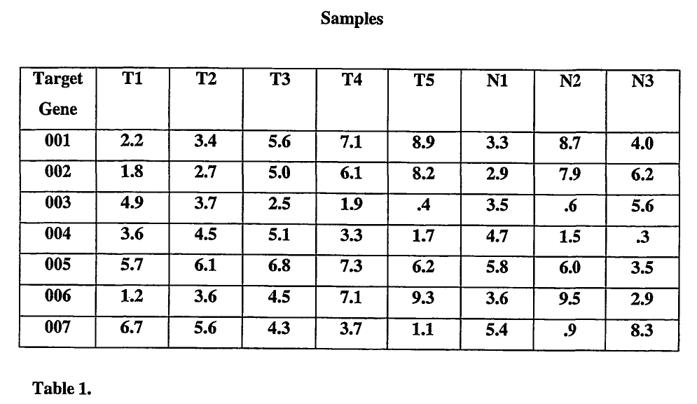

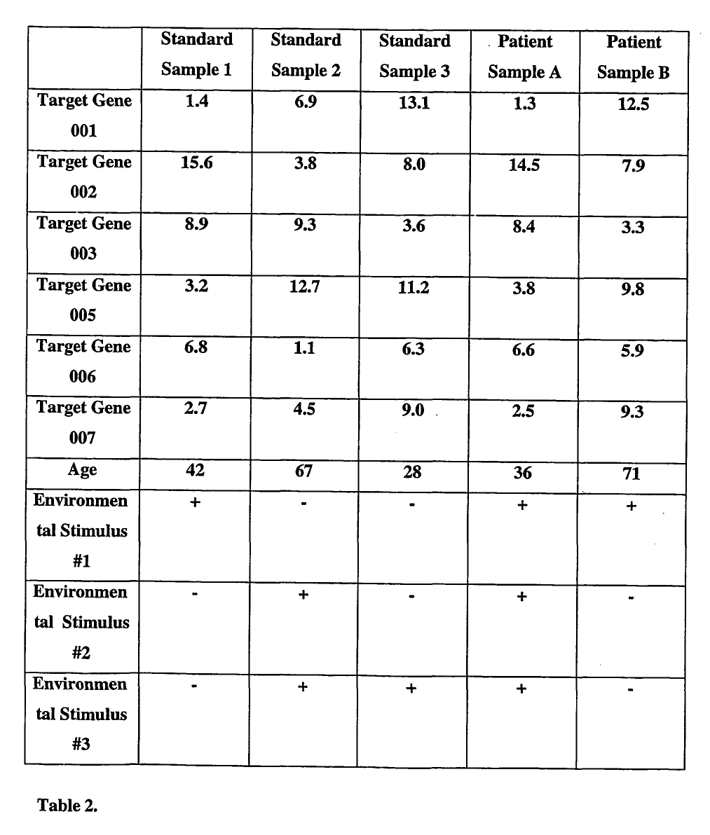

The biochemical data gleaned from the identification of interacting molecules with transcription factor target proteins is of unparalleled value for the development of agents of therapeutic intervention. By focusing on the biochemical events downstream of a particular transcription factor it is possible to circumvent effects on undesired cellular cascades and design drugs which will exhibit higher efficacy and fewer side effects than those which are currently available. FIG. 7 illustrates a theoretical example of the process for the identification of a therapeutic compound for the treatment of cancer through the implementation of transcription factor target protein array technology. A transcription factor target protein array representing targets for the tumor suppressor p53 is tested against a fluorescent tag conjugated small molecule for interactions between the molecule and particular p53 target proteins. Fluorescent light emission reveals a specific binding interaction between the small molecule and what is determined to be a G protein coupled receptor (GPCR) thought to inhibit tumorigenesis (for review see Gershengom et al. 2001, Endocrinology, 142: 2-10). Tissue culture experiments are subsequently conducted to determine a putative negative or positive effect of the small molecule drug on the receptor's ability to transmit signals intracellularly to ultimately affect cellular proliferative and apoptotic events. If the small molecule is determined to inhibit receptor function antagonists are designed hamper inactivation of the receptor thus driving constitutive receptor function. If the small molecule is revealed to activate the receptor and thereby promote inhibition of tumorigenesis further analogs are developed to optimize interaction specificities and increase the activation state of the receptor. In both cases it is possible to develop potential therapeutic agents superior to existing treatment strategies, the focus of which is directed at transcription factor target proteins in vivo.

### 7.0 REFERENCES

### Patent Documents

Hudson et al., U.S. Pat. No. 5,591,646, Issued January, 1997
Buettner et al., U.S. Pat. No. 5,834,318, Issued November, 1998
Lockhart et al., U.S. Pat. No. 6,040,138, Issued March, 2000
Burgess et al., PCT application #PCT/US01/24823, filed Aug. 14, 2000
McCasky et al., U.S. Pat. No. 6,100,030, Issued August, 2000
Leighton et al., U.S. Pat. No. 6,136,592, Issued October, 2000
Schatz et al., U.S. Pat. No. 6,156,511, Issued December, 2000

### Other References

Benashski et al., Methods, 2000, 22: 365-371
Chang et al., 1998, Clinical Immunology and Immunopathology, 89(1): 71-8
Chen et al., 1996, Developmental Genetics, 19(2): 119-30
Debouck et al., 1999, Nature Genetics Supplement, 21: 48-50
DeRisi et al., Science, 278: 680-686
Drysdale et al., 2000, Yeast, 17(2):159-66
Dunaief et al., 1994, Cell, 79(1):119-30
E1-Diery et al., 1993, Cell, 75: 817-825
Emili et al., 2000, Nature Biotechnology, 18: 393-397
Gershengorn et al., 2001, Endocrinology, 142: 2-10
Gygi et al., 1999, Nature Biotechnology, 17: 994-999
Herzig et al., 1997, Proceedings of the National Academy of Sciences, 94: 7543-7548
Hufton et al., 1999, J. Immunol. Methods, 231: 39-51
Hughes et al., 2000, Cell, 102: 109-126
Lamond et al., 1997, Trends Cell Biol., 7: 139-142
Levine et al., 1991, Nature, 351: 453456
MacBeath et al., 2000, Science, 289: 1760-1763
Martzen et al., 1999, Science, 286: 1153-1155
Moroy et al., 2000, Cellular and Molecular Life Sciences, 57(6): 957-75
Neubauer et al., 1997, Proc. Natl. Acad. Sci. USA, 94: 385-390
Nichogiannopoulou et al., 1998 Seminars in Immunology, 10: 119-125
Ochman et al., 1988, Genetics, 120(3): 621-623
Rhodes et al., 1994, Current Opinions in Genetic Development, 4: 709-717
Solomon et al., 1988, Cell, 53: 937-947
Speliman et al., 1998, Cell, 9: 3273-3297
Tenbaum et al., 1997, International Journal of Biochemistry and Cell Biology, 29: 1325-1341
Tjian and Maniatis, 1994, Cell, 77: 5-8
Uetz et al., 2000, Nature, 403: 623-627
Venter et al., 2001, Science, 291: 1304-1351
Zozulya et al., 1999, Nature Biotechnol., 17: 1193-1198
Zweiger et al., 1997, Trends in Biotechnology, 17: 429436

1. A method according to the present invention which utilizes modified sequential chromosomal immunoprecipitation and cloning procedures for the discovery of transcription factor target genes from cells whereby said genes are organized into an array format.

2. A method according to claim 1 comprising the process of:
a) cross-linking protein/DNA complexes in cells or tissues;
b) immunoprecipitating said protein/DNA complexes with antibodies which recognize transcription factors;
c) purifying DNA present within immunoprecipitated protein/DNA samples;
d) organizing said purified DNA sequences into an array format.

3. A method according to claim 2 in which said purification of DNA present within immunoprecipitated protein/DNA samples includes amplification via inverse polymerase chain reaction (I-PCR) utilizing oligonucleotides corresponding to transcription factor binding sites to determine flanking nucleotide sequences present within discovered DNA fragments.

4. A method according to claim 2 in which said arrays consist of DNA templates bound to solic supports, for purposes of assessing the expression patterns or levels of transcription factor target genes.

5. A method according to claim 4 in which said transcription factor target genes consist of transcribed sequences, including coding sequences which correspond to amino acid composition.

6. A method according to claim 2 in which purified DNA fragments are utilized to cross hybridize against libraries of DNA sequences for the purposes of creating transcription factor target

7. An antibody according to claim 2 whereby said antibody allows for the purification of protein/protein and/or protein/DNA complexes from cells, for purposes of creating arrays and/or microarrays of transcription factor target genes.

8. A protein/DNA complex isolated from cells according to claim 2 whereby said protein DNA/complex results in the identification of transcription factor target genes, for purposes of constructing arrays and/or microarrays of said target genes.

9. DNA fragments isolated from protein/DNA complexes according to claim 8 whereby said DNA fragments encode transcription factor target genes, for purposes of constructing arrays and/or microarrays of said target genes.

10. Nucleotide sequences present in DNA fragments isolated according to methods described in claim 2 wherein said sequences represent transcription factor target genes and are utilized for purposes of constructing arrays of said sequences.

11. Arrays of transcription factor target gene sequences, for purposes of monitoring the expression patterns of transcription factor targets in given samples.

12. A method according to claim 2 which further comprises the process of translating isolated transcription factor target gene sequences for the purposes of constructing target protein arrays.

13. A method according to claim 12 in which said arrays are of a chemical/“nonliving” nature or biological/“living” nature.

14. Arrays of transcription factor target proteins as described in claim 13.

15. A transcription factor target protein/protein interaction complex identified by arrays described in claim 14 in which said protein/protein complex represents the interaction between transcription factor target protein sequences and other protein sequences, for the purposes of characterizing transcription factor target protein interacting molecules.

16. A transcription factor target protein/small molecule complex identified by arrays described it claim 14 in which said protein/small molecule complex represents the interaction between transcription factor target protein sequences and small molecules, for the purposes of characterizing transcription factor target protein interacting molecules.

17. A transcription factor target protein/metal complex identified by arrays described in claim 14 in which said protein/metal complex represents the interaction between transcription factor target protein sequences and charged or uncharged metals, for the purposes of characterizing transcription factor target protein interacting molecules.

18. A transcription factor target protein/nucleotide sequence complex identified by arrays described in claim 14 in which said protein/nucleotide sequence complex represents the interaction between transcription factor target protein sequences and nucleotide sequences of DNA or RNA origin, for the purposes of characterizing transcription factor target protein interacting molecules.

19. Proteins which are discovered as specifically interacting with transcription factor target protein sequences through the use of arrays according to claim 14.

20. Metals which are discovered as specifically interacting with transcription factor target proteii sequences through the use of arrays described by claim 14.

21. Nucleotide sequences which are discovered as specifically interacting with transcription factor target protein sequences through the use of arrays described in claim 14.

22. Simple sugars and oligosaccharides which are discovered as specifically interacting with transcription factor target protein sequences through the use of arrays described by claim 14.

23. Therapies designed as a result of the knowledge obtained from the discovery of interactions between transcription factor target protein sequences and proteins, amino acid or peptide sequences, nucleotide sequences, small molecules, metals, simple sugars and oligosaccharides through the use of arrays according to claim 14.
